(19) United States
(12) Patent Application Publication
(10) Pub. No.: US 2007/0259874 A1
(43) Pub. Date: Nov. 8, 2007

(54) Phosphodiesterase Inhibitors

(76) Inventors: Venkata P. Palle, Haryana (IN); Sarala Balachandran, Delhi (IN); Nidhi Gupta, Delhi (IN); Gagan Kukreja, Delhi (IN); Manoj Kumar Khera, Haryana (IN); Lalit Kumar Baregama, Rajasthan (IN); Raghuramaiah Mandadapu, Andhra Pradesh (IN); Abhijit Ray, Delhi (IN); Sunanda Ghose Dastidar, Delhi (IN)

Correspondence Address: RANBAXY INC., 600 COLLEGE ROAD EAST, SUITE 2100, PRINCETON, NJ, 08540, US

(21) Appl. No.: 10/596,059

(22) PCT Filed: Nov. 26, 2004
(86) PCT No.: PCT/IB04/03893

§ 371 Date: Apr. 16, 2007

Related U.S. Application Data
(60) Provisional application No. 60/525,347, filed on Nov. 26, 2003.

(51) Int. Cl. A61K 31/42; A61K 31/4245; A61K 31/433; A61K 31/5375; A61P 1/04; A61P 11/08; A61P 17/00; A61P 17/06; A61P 19/02; A61P 29/00; A61P 37/00; A61P 37/08; A61P 43/00; A61P 9/00; C07D 261/00; C07D 271/06; C07D 271/10; C07D 271/113; C07D 285/135; C07D 413/00

(52) U.S. Cl. 514/236.8; 514/363; 514/364; 544/137; 548/131; 548/132; 548/138; 548/143; 548/243

(57) ABSTRACT

The present invention relates to isoxazoline derivatives and their analogues, which can be used as phosphodiesterase (PDE) type IV selective inhibitors. Compounds disclosed herein can be useful in the treatment of AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases, especially in humans. Processes for the preparation of disclosed compounds, pharmaceutical compositions containing the disclosed compounds, and their use as PDE type IV selective inhibitors, are provided.

### FIELD OF THE INVENTION

The present invention relates to isoxazoline derivatives and their analogues, which can be used as phosphodiesterase (PDE) type IV selective inhibitors. Compounds disclosed herein can be useful in the treatment of AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans. Processes for the preparation of disclosed compounds, pharmaceutical compositions containing the disclosed compounds, and their use as PDE type IV selective inhibitors, are provided.

### BACKGROUND OF THE INVENTION

It is known that cyclic adenosine-3′,5′-monophosphate (cAMP) exhibits an important role of acting as an intracellular secondary messenger (E. W. Sutherland, and T. W. Roll Pharmacol. Rev, 1960, 12, 265). Its intracellular hydrolysis to adenosine 5′-monophosphate (AMP) causes number of inflammatory conditions which are not limited to psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis. The most important role in the control of cAMP (as well as of cGMP) level is played by cyclic nucleotide phosphodiesterases (PDE) which represents a biochemically and functionally, highly variable superfamily of the enzyme; eight distinct families with more than 15 gene products are currently recognized. Although PDE I, PDE II, PDE III, PDE IV, and PDE VII all use cAMP as a substrate, only PDE IV and PDE VII are highly selective for hydrolysis of cAMP. Inhibitors of PDE, particularly the PDE IV inhibitors, such as rolipram or Ro-1724 are therefore known as cAMP-enhancers. Immune cells contain type IV and type III PDE, the PDE IV type being prevalent in human mononuclear cells. Thus the inhibition of phosphodiesterase type IV has been a target for modulation and, accordingly, for therapeutic intervention in a range of disease processes.

The initial observation that xanthine derivatives, theophylline and caffeine inhibit the hydrolysis of cAMP led to the discovery of the required hydrolytic activity in the cyclic nucleotide phosphodiesterase (PDE) enzymes. More recently, distinct classes of PDE have been recognized (J. A. Bervo and D. H. Reifsnyder, TIPS, 1990, 11, 150), and their selective inhibition has led to improved drug therapy (C. D. Nicholus, R. A. Challiss and M. Shahid, TIPS, 1991, 12, 19). Thus it was recognized that inhibition of PDE IV could lead to inhibition of inflammatory mediator release (M. W. Verghese et. al, J. Mol. Cell. Cardiol. 1989, 12 (Suppl.II), S 61) and airway smooth muscle relaxation.

U.S. Pat. No. 5,686,434 (National stage of WO 95/14680) discloses 3-aryl-2-isoxazolines as anti-inflammatory agents. U.S. Pat. Nos. 6,114,367 and 5,869,511 (National stage of WO 95/24398) disclose isoxazoline compounds as inhibitors of TNF release. WO 95/14681 discloses a series of isoxazoline compounds as anti-inflammatory agents. WO 02/100332 discloses isoxazoline compounds having macrophage inhibitory factor (MIF) antagonist activity.

### SUMMARY OF THE INVENTION

The present invention provides isoxazoline derivatives and their analogues, which can be used for the treatment of AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases, and the processes for the synthesis of these compounds.

Pharmaceutically acceptable salts, pharmaceutically acceptable solvates, enantiomers, diastereomers or N-oxides of these compounds having the same type of activity are also provided.

Pharmaceutical compositions containing the compounds, which may also contain pharmaceutically acceptable carriers or diluents, can be used for the treatment of AIDS, asthma, arthritis, bronchitis, chronic obstructive pulmonary disease (COPD), psoriasis, allergic rhinitis, shock, atopic dermatitis, Crohn's disease, adult respiratory distress syndrome, eosinophilic granuloma, allergic conjunctivitis, osteoarthritis, ulcerative colitis and other inflammatory diseases.

Other aspects will be set forth in the accompanying description which follows and in part will be apparent from the description or may be learnt by the practice of the invention.

In accordance with one aspect, there are provided compounds having the structure of Formula I: 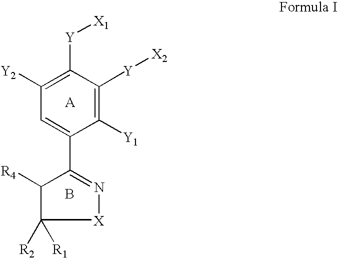
their pharmaceutically acceptable salts, pharmaceutically acceptable solvates, enantiomers, diastereomers or N-oxides.
When X is oxygen in Formula I:
R₁ can represent: hydrogen; alkyl; alkenyl; alkynyl; cycloalkyl; cyano; nitro; amino; substituted amino; hydroxyl; alkoxy; aryloxy; COR′ or COOR′ (wherein R′ can be hydrogen, alkyl, alkenyl, alkynyl, (un)saturated cycloalkyl, aryl, aralkyl, heterocyclyl, (heterocyclyl)alkyl, or (heteroaryl)alkyl); aryl; aralkyl; heteroaryl; heterocyclyl; (heteroaryl) alkyl; (heterocyclyl) alkyl; (CH₂)₁₋₄OR′ (wherein R′ is as defined above, but also including hydroxy); C(═O)NRₓR_{y} (wherein Rₓ and R_{y} can be independently selected from hydrogen, alkyl, C₃₋₆ alkenyl, C₃₋₆ alkynyl, (un)saturated cycloalkyl, aryl, aralkyl, heteroaryl, heterocyclyl, heteroarylalkyl, or heterocyclylalkyl); or (CH₂)ₘ—C(═O)R₃ [wherein m is an integer in the range of 0-2 and R₃ can be optionally substituted Rₚ or R_{q} (wherein Rₚ can be a 4-12 membered (un)saturated monocyclic or bicyclic ring containing 1-4 heteroatom(s) selected from N, O and S wherein the ring can be attached to (CH₂)ₘC(═O) through N and R_{q} can be a 4-12 membered (un)saturated monocyclic or bicyclic ring containing 0-4 heteroatom(s) selected from the group consisting of N, O and S wherein the ring can be attached to (CH₂)ₘC(═O) through C) and wherein the substituents of R₃ can be one or more of: alkyl, alkenyl, alkynyl, (un)saturated cycloalkyl, halogen, hydroxyl alkoxy, aryloxy, nitro, cyano, amino, substituted amino, hydroxyalkyl, oxo, acyl, optionally substituted amino (wherein the substituents are selected from C₁-C₆ alkyl, aryl, aralkyl, or cycloalkyl), aryl, carboxyl, alkaryl, carbamoyl, alkyl ether, C(═O)NR₅R₆ (wherein R₅ and R₆ are independently selected from hydrogen, alkyl, C₃₋₆ alkenyl, C₃₋₆ alkynyl, aryl, and aralkyl), optionally substituted monocyclic or bicyclic 4-12 membered carbocyclic ring system (wherein the optional substituent(s) is/are selected from alkyl, alkenyl, alkynyl, halogen, hydroxyl, and alkoxy), heteroaryl, heterocyclyl, heteroarylalkyl, or heterocyclylalkyl].
R₂ can represent: cyano; heteroaryl; heterocyclyl; or (CH₂)ₙNHCOR₇ (wherein n represents an integer 1 to 6 and R₇ can represent hydrogen, alkyl, alkenyl, alkynyl, (un)saturated, cycloalkyl, alkoxy, aryloxy, aryl, aralkyl, heteroaryl, heterocyclyl, (CH₂)₁₋₄OR′ wherein R′ is the same as defined above, or NRₓR_{y} wherein Rₓ and R_{y} are the same as defined above).
R₄ can represent: hydrogen; alkyl; halogen; cyano; carboxy; or C(═O)NRₓR_{y} wherein Rₓ and R_{y} are the same as defined above.
X₁ and X₂ can be independently selected from: hydrogen; alkyl; alkenyl; alkynyl; cycloalkyl; acyl; aryl; aralkyl; heteroaryl; heterocyclyl; (heteroaryl)alkyl; or (heterocyclyl)alkyl.
Y can represent: an oxygen atom; a sulphur atom; or NR (wherein R is selected from hydrogen, alkyl, alkenyl, alkynyl, un(saturated) cycloalkyl, acyl, aryl, aralkyl, heteroaryl, heterocyclyl, (heteroaryl)alkyl, or (heterocyclyl)alkyl).
Y₁ and Y₂ can be independently selected from: hydrogen; alkyl; nitro; cyano; halogen; OR wherein R is the same as defined earlier; SR wherein R is the same as defined earlier; NHR wherein R is the same as defined earlier; COOR′; or COR′ wherein R′ is the same as defined above.
Further, Y₁ and X₂, X₁ and Y₂, X₁ and X₂ may together form a cyclic ring fused with the ring A containing 3-5 carbon atoms within the ring and having 1-3 heteroatoms selected from N, O or S.
When X is NR₈ or S wherein R is hydrogen, lower alkyl (C₁-C₆) or aryl:
R₁, R₄, X₁, X₂, Y, Y₁ and Y₂ are the same as defined above.
R₂ can represent: (CH)ₙNHCOR₇ (wherein n represents an integer 1 to 6 and R₇ is the same as defined above), with the provisio that when R₂ is heterocyclyl, R₁ can not be (CH₂)₁₋₄OR′, C(═O)NRₓR_{y} or (CH₂)ₘ—C(═O)R₃.

In one particular embodiment, there are provided compounds having the structure of Formula XXXII, 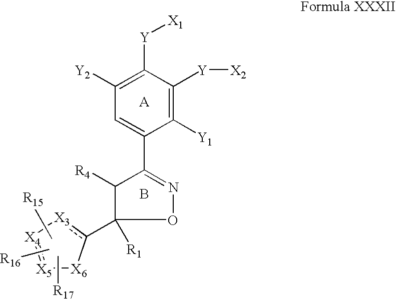
their pharmaceutically acceptable salts, pharmaceutically acceptable solvates, enantiomers, diastereomers or N-oxides.
In such compounds of Formula XXXII,
Y, Y₁, Y₂, R₁ and R₄ can be as defined for Formula I;
X₁ can be alkyl;
X₂ can be alkyl, cycloalkyl, or aralkyl;
X₃, X₄, X₅ and X₆ can be independently selected from C, CH, CH₂, CO, CS, NH, N, O and S;
R₁₅, R₁₆, and R₁₇ can be independently selected from no atom, alkyl, COCH₃, COOC₂H₅, NH₂, NH-cyclopropyl, CN and SH; and
------ represents an optional double bond.

In another embodiment, there are provided compounds having the structure of Formula XXXIII, 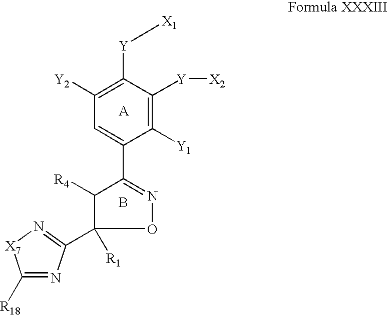
their pharmaceutically acceptable salts, pharmaceutically acceptable solvates, enantiomers, diastereomers or N-oxides.
In such compounds of Formula XXXIII,
Y, Y₁, Y₂, X₁, X₂, R₁ and R₄ can be as defined for Formula I;
X₇ can be O or S; and
R₁₈ can represent hydrogen, alkyl, aryl, heteroaryl, cycloalkyl, or heterocyclyl.

In yet another embodiment, there are provided compounds having the structure of Formula XXXIV,

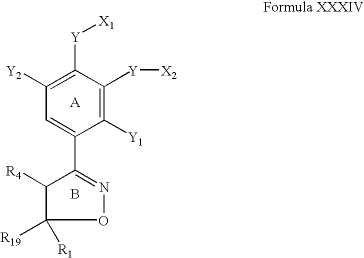

their pharmaceutically acceptable salts, pharmaceutically acceptable solvates, enantiomers, diastereomers or N-oxides.

In such compounds of Formula XXXV,

Y, Y₁, Y₂, X₁, X₂, R₁ and R₄ can be as defined for Formula I; and

R₁₉ can represent cyano, —CONHNH₂, —C(NH₂)═N—O—C(O)R′, (CH₂)ₙNHCOR₇ or 6-membered heteroaryl, wherein R₁, R₇ and n are the same as defined for Formula I.

The following definitions apply to terms as used herein:

The term “alkyl,” unless otherwise specified, refers to a monoradical branched or unbranched saturated hydrocarbon chain having from 1 to 20 carbon atoms. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, —NHC(═O)R_{f}, —NR_{f}R_{q}, —C(═O)NR_{f}R_{q}, —NHC(═O)NR_{f}R_{q,}, —C(═O)heteroaryl, C(═O)heterocyclyl, O—C(═O)NR_{f}R_{q} {wherein R_{f} and R_{q} are independently selected from alkyl, alkenyl, cycloalkyl, cycloalkenyl, aryl, aralkyl, heterocyclyl, heteroaryl, heterocyclylalkyl, heteroarylalkyl}, nitro, or —SO₂R₆ (wherein R₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, carboxy, —NR_{f}R_{q}, —C(═O)NR_{f}R_{q}, —OC(═O)NR_{f}R_{q}, —NHC(═O)NR_{f}R_{q} (wherein R_{f} and R_{q} are the same as defined earlier), hydroxy, alkoxy, halogen, CF₃, cyano, and —SO₂R₆, (wherein R₆ are the same as defined earlier); or an alkyl group also may be interrupted by 1-5 atoms of groups independently selected from oxygen, sulfur or —NRₐ— {wherein Rₐ is selected from hydrogen, alkyl, cycloalkyl, alkenyl, cycloalkenyl, alkynyl, aryl, acyl, aralkyl, —C(═O)OR_{f} (wherein R_{f} is the same as defined earlier), SO₂R₆ (where R₆ is as defined earlier), or —C(═O)NR_{f}R_{q} (wherein R_{f} and R_{q} are as defined earlier)}. Unless otherwise constrained by the definition, all substituents may be substituted further by 1-3 substituents selected from alkyl, carboxy, —NR_{q}, —C(═O)NR_{f}R_{q}, —O—C(═O)NR_{f}R_{q} (wherein R_{f} and R_{q} are the same as defined earlier) hydroxy, alkoxy, halogen, CF₃, cyano, and —SO₂R₆ (where R₆ is same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans, or geminal geometry. In the event that alkenyl is attached to a heteroatom, the double bond cannot be alpha to the heteroatom. Alkenyl groups may be substituted further with one or more substituents selected from alkyl, alkynyl, alkoxy, cycloalkyl, cycloalkenyl, acyl, acylamino, acyloxy, —NHC(═O)R_{f}, —NR_{f}R_{q}, —C(═O)NR_{f}R_{q}, —NHC(═O)NR_{f}R_{q}, —O—C(═O)NR_{f} (wherein R_{f} and R_{q} are the same as defined earlier), alkoxycarbonylamino, azido, cyano, halogen, hydroxy, oxo, thiocarbonyl, carboxy, arylthio, thiol, alkylthio, aryl, aralkyl aryloxy, heterocyclyl, heteroaryl, heterocyclyl alkyl, heteroaryl alkyl, aminosulfonyl, aminocarbonylamino, alkoxyamino, nitro, or SO₂& (wherein R₆ are is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NR_{f}R_{q}, —C(═O)NR_{f}R_{q}, —O—C(═O)NR_{f}R_{q} (wherein R_{f} and R_{q} are the same as defined earlier) and —SO₂R₆ (where R₆ is same as defined earlier).

The term “alkynyl,” unless otherwise specified, refers to a monoradical of an unsaturated hydrocarbon, having from 2 to 20 carbon atoms. In the event that alkynyl is attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, aminocarbonylamino, nitro, heterocyclyl, heteroaryl, heterocyclylalkyl, heteroarylalkyl, —NHC(═O)R_{f}, —NR_{f}R_{q}, —NHC(═O)NR_{f}R_{q}, —C(═O)NR_{f}R_{q}, —O—C(═O)NR_{f}R_{q} (wherein R_{f} and R_{q} are the same as defined earlier), or —SO₂R₆ (wherein R₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NR_{f}R_{q}, —C(═O)NR_{f}R_{q}, —NHC(═O)NR_{f}R_{q}, —C(═O)NR_{f}R_{q} (wherein R_{f} and R_{q} are the same as defined earlier), cyano, or —SO₂R₆ (where R₆ is same as defined earlier).

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl and bicyclo[2.2.1]heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{f}R_{q}, —NHC(═O)NR_{f}R_{q}, —NHC(═O)R_{f}, —C(═O)NR_{f}R_{q}, —O—C(═O)NR_{f}R_{q} (wherein R_{f} and R_{q} are the same as defined earlier), nitro, heterocyclyl, heteroaryl, heterocyclylalkyl, heteroarylalkyl, or SO₂—R₆ (wherein R₆ is same as defined earlier). Unless otherwise constrained by the definition, cycloalkyl substituents optionally may be substituted further by 1-3 substituents selected from alkyl, carboxy, hydroxy, alkoxy, halogen, CF₃, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), cyano or —SO₂R₆ (where R₆ is same as defined earlier).

The term “alkoxy” denotes the group O-alkyl, wherein alkyl is the same as defined above.

The term “aryl,” unless otherwise specified, refers to carbocyclic aromatic groups, for example, phenyl, biphenyl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₑ (wherein Rₑ is hydrogen, alkyl, alkenyl, cycloalkyl, aralkyl, heterocyclylalkyl, heteroarylalkyl), NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), —SO₂R₆ (wherein R₆ is same as defined earlier), carboxy, heterocyclyl, heteroaryl, heterocyclylalkyl, heteroarylalkyl or amino carbonyl amino. The aryl group optionally may be fused with a cycloalkyl group, wherein the cycloalkyl group may optionally contain heteroatoms selected from O, N or S.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl and the like.

The term “aralkenyl,” unless otherwise specified, refers to alkenyl-aryl linked through alkenyl (wherein alkenyl is as defined above) portion and the alkenyl portion contains 1 to 6 carbon atoms and aryl is as defined below.

The term “aryloxy” denotes the group O-aryl, wherein aryl is as defined above.

The term “carboxy,” as defined herein, refers to —C(═O)OH.

The term “heteroaryl,” unless otherwise specified, refers to an aromatic ring structure containing 5 or 6 ring atoms, or a bicyclic aromatic group having from 8 to 10 ring atoms, with one or more heteroatom(s) independently selected from N, O or S optionally substituted with 1 to 4 substituent(s) selected from halogen (e.g. F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{f}R_{q}, CH═NOH, —(CH₂)_{w}C(═O)R_{g} {wherein w is an integer from 0-4 and R_{g} is hydrogen, hydroxy, OR_{f}, NR_{f}R_{q}, —NHOR_{z} or —NHOH}, —C(═O)NR_{f}R_{q}, and —NHC(═O)NR_{f}R_{q}, —SO₂R₆, —O—C(═O)NR_{f}R_{q}, —O—C(═O)R_{f}, —O—C(═O)OR_{f} (wherein R₆, R_{f} and R_{q} are as defined earlier, and R_{g} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzothiazolyl, or benzoxazolyl, and the like.

The term ‘heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, aryl, alkoxy, alkaryl, cyano, nitro, oxo, carboxy, heterocyclyl, heteroaryl, —O—C(O)R_{f}, —O—C(═O)OR_{f}, —C(═O)NR_{f}R_{q}, SO₂R₆, —O—C(═O)NR_{f}R_{q}, —NHC(═O)NR_{f}R_{q}, —NR_{f}R_{q} (wherein R₆, R_{f} and R_{q} are as defined earlier) or guanidine. Heterocyclyl can optionally include rings having one or more double bonds. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, dihydropyridinyl, dihydroisoxazolyl, dihydrobenzofuryl, azabicyclohexyl, dihydroindolyl, pyridinyl, isoindole 1,3-dione, piperidinyl or piperazinyl.

“Heteroarylalkyl” refers to alkyl-heteroaryl group linked through alkyl portion, wherein the alkyl and heteroaryl are as defined earlier.

“Heterocyclylalkyl” refers to alkyl-heterocyclyl group linked through alkyl portion, wherein the alkyl and heterocyclyl are as defined earlier.

“Acyl” refers to —C(═O)R″ wherein R″ is selected from hydrogen, alkyl, cycloalkyl, aryl, aralkyl, heteroaryl, heterocyclyl, heteroarylalkyl or heterocyclylalkyl.

“Alkylcarbonyl” refers to —C(═O)R″, wherein R″ is selected from alkyl, cycloalkyl, aryl, aralkyl, heteroaryl, heterocyclyl, heteroarylalkyl or heterocyclylalkyl.

“Alkylcarboxy” refers to C(═O)R″, wherein R″ is selected from alkyl, cycloalkyl, aryl, aralkyl, heteroaryl, heterocyclyl, heteroarylalkyl or heterocyclylalkyl.

“Amine,” unless otherwise specified, refers to —NH₂. “Substituted amine,” unless otherwise specified, refers to —N(Rₖ)₂, wherein each Rₖ independently is selected from hydrogen {provided that both Rₖ groups are not hydrogen (defined as “amino”)}, alkyl, alkenyl, alkynyl, aralkyl, cycloalkyl, aryl, heteroaryl, heterocyclyl, heterocyclylalkyl, heteroarylalkyl, acyl, SO₂R₆ (wherein R₆ is as defined above), —C(═O)NR_{f}R_{q}, NHC(═O)NR_{f}R_{q}, or —NHC(═O)OR_{f} (wherein R_{f} and Rₑ are as defined earlier).

“Thiocarbonyl” refers to —C(═S)H. “Substituted thiocarbonyl” refers to —C(═S)R″, wherein R″ is selected from alkyl, cycloalkyl, aryl, aralkyl, heteroaryl, heterocyclyl, heteroarylalkyl or heterocyclylalkyl, amine or substituted amine.

Unless otherwise constrained by the definition, all substituents optionally may be substituted further by 1-3 substituents selected from alkyl, aralkyl, cycloalkyl, aryl, heteroaryl, heterocyclyl, carboxy, carboxyalkyl, hydroxy, alkoxy, halogen, CF₃, cyano, —C(=T)NR_{f}R_{q}, —O(C═O)NR_{f}R_{q} (wherein R_{f}, R_{q} and T are the same as defined earlier) and —OC(=T)NR_{f}R_{q,}, —SO₂R₆ (where R₆ is the same as defined earlier).

The term “leaving group” refers to groups that exhibit or potentially exhibit the properties of being labile under the synthetic conditions and also, of being readily separated from synthetic products under defined conditions. Examples of leaving groups include, but are not limited to, halogen (e.g., F, Cl, Br, I), triflates, tosylate, mesylates, alkoxy, thioalkoxy, or hydroxy radicals and the like.

The term “protecting groups” refers to moieties that prevent chemical reaction at a location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule.

The term “pharmaceutically acceptable salts” refers to derivatives of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like.

The compounds provided herein can be used for treating AIDS, asthma, arthritis, bronchitis, chronic obstructive pulmonary disease, psoriasis, allergic rhinitis, shock, atopic dermatitis, Crohn's disease, adult respiratory distress syndrome, eosinophilic granuloma, allergic conjunctivitis, osteoarthritis, ulcerative colitis and other inflammatory diseases.

In accordance with yet another aspect, there are provided processes for the preparation of the compounds as described herein.

### DETAILED DESCRIPTION OF THE INVENTION

The compounds described herein may be prepared by techniques well known in the art and familiar to the average synthetic organic chemist. In addition, the compounds of present invention may be prepared by the following reaction sequences as depicted in schemes I, IA, IB, II, III, IV and V.

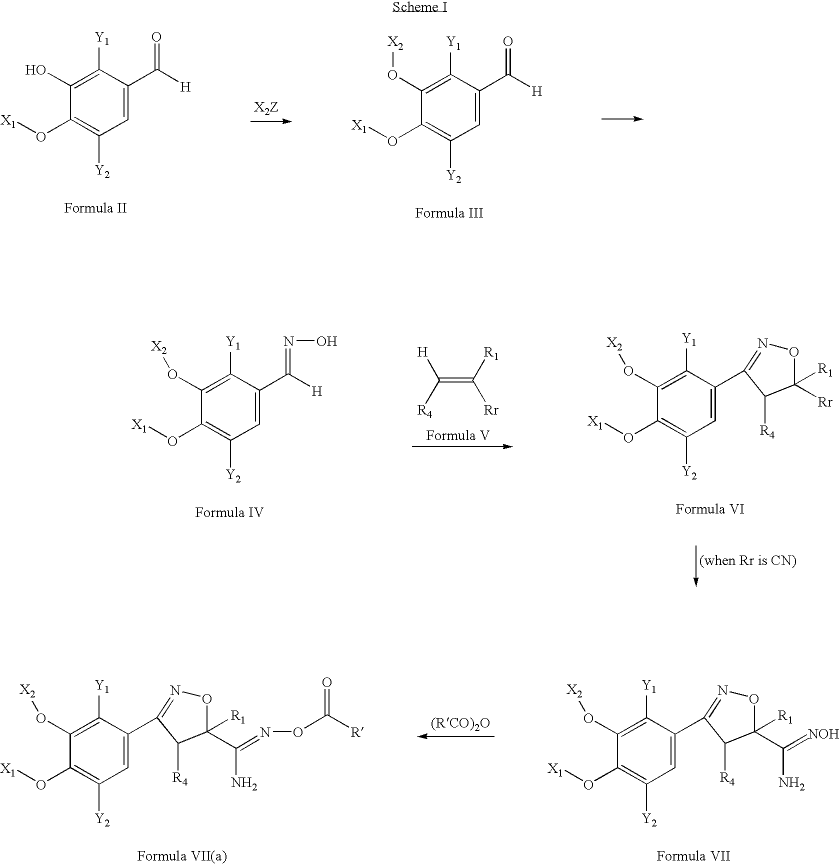

The compounds of Formula VII (a) can be prepared according to Scheme I. Thus, reacting a compound of Formula II with compound of Formula X₂Z (wherein Z is halogen) to give a compound of Formula III [wherein X₁, X₂ (except hydrogen), Y₁ and Y₂ are the same as defined earlier], which on reaction with hydroxylamine hydrochloride gives a compound of Formula IV, which on treatment with a compound of Formula V gives a compound of Formula VI (wherein R₁ and R₄ are the same as defined earlier and Rr represents [(CH₂)ₙCN, COOH, COOCH₃, CHO or pyridyl, wherein n is 0 to 2)], which on reaction with hydroxylamine hydrochloride (when Rr is CN) to give a compound of Formula VII, which is finally reacted with a compound of Formula (R′CO)₂O to give a compound of Formula VII(a) (wherein R′ is the san as defined earlier).

The reaction of a compound of Formula II with a compound of Formula X₂Z to give a compound of Formula III can be carried out in a solvent, for example, tetrahydrofuran, dimethylformamide, dimethylsulphoxide or acetonitrile.

The reaction of a compound of Formula II with compound of Formula X₂Z can be carried out in the presence of potassium iodide and an inorganic base, for example, sodium carbonate, sodium bicarbonate, potassium carbonate or potassium bicarbonate.

The reaction of a compound of Formula III with hydroxylamine hydrochloride to give a compound of Formula IV can be carried out in the presence of sodium acetate or potassium acetate in a solvent, for example, methanol, ethanol, propanol or n-butanol.

The reaction of a compound of Formula IV with a compound of Formula V to give a compound of Formula VI can be carried out in the presence of sodium hypochlorite in a solvent, for example, tetrahydrofuran, dimethylformamide, dimethylsulphoxide or acetonitrile.

The reaction of a compound of Formula VI with hydroxylamine hydrochloride to give a compound of Formula VII can be carried out in a solvent, for example, tetrahydrofuran, dimethylformamide, dimethylsulphoxide, acetonitrile, acetone, ethanol or mixtures thereof.

The reaction of a compound of Formula VI with hydroxylamine hydrochloride can be carried out in the presence of an inorganic base, for example, sodium carbonate, sodium bicarbonate, potassium carbonate or potassium bicarbonate.

The reaction of a compound of Formula VII with a compound of Formula (R′CO)₂O to give a compound of Formula VII (a) can be carried out in a solvent, for example, tetrahydrofuran, dimethylformamide, dimethylsulphoxide or acetonitrile.

The reaction of a compound of Formula VII with a compound of Formula (R′CO)₂O can be carried out in the presence of an organic base, for example, trimethylamine, triethylamine or pyridine.

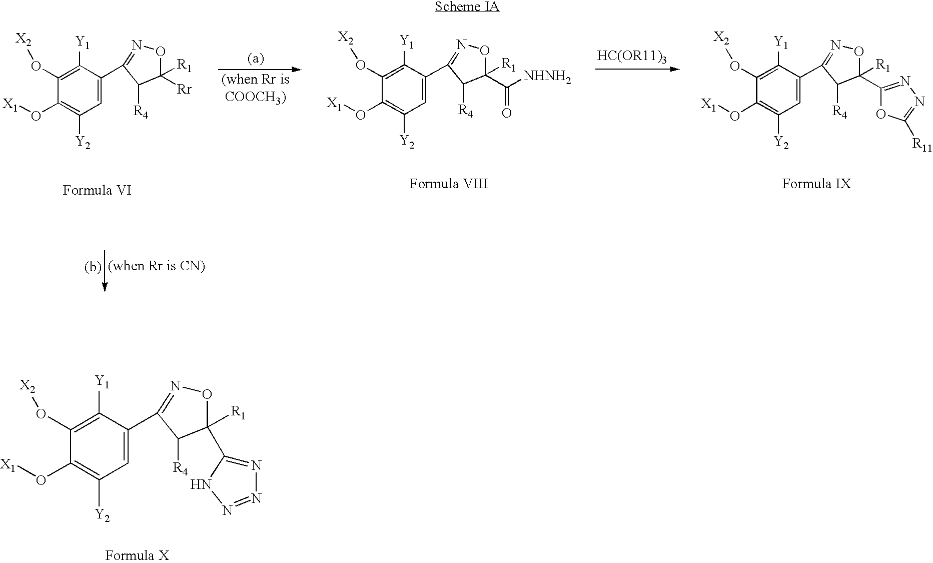

The compounds of Formula IX and X can be prepared according to scheme IA. Thus, reacting a compound of
(a) Formula VI (when Rr is COOCH₃) with hydrazine hydrate to give a compound of Formula VIII (wherein X₁, X₂, Y₁, Y₂, R₁ and R₄ are the same as defined earlier), which on reaction with a compound of Formula HC(OR₁₁)₃ gives a compound of Formula IX (wherein R₁₁ represents alkyl from C₁ to C₃); or
(b) Formula VI (when Rr is CN) with sodium azide to give a compound of Formula X (wherein X₁, X₂, Y₁, Y₂, R₁ and R₄ are the same as defined earlier),

The reaction of a compound of Formula VI with hydrazine hydrate to give a compound of Formula VIII can be carried out at a temperature ranging, for example, from 120 to 140° C.

The reaction of a compound of Formula Vm with a compound of Formula HC(OR₁₁)₃ to give a compound of Formula IX can be carried out at a temperature ranging, for example, from 120 to 160° C.

The reaction of a compound of Formula VI with sodium azide to give a compound of Formula X can be carried out in a solvent, for example, benzene, toluene or xylene.

The reaction of a compound of Formula VI with sodium azide to give a compound of Formula X can be carried out in the presence of hydrochloride salt of an organic base, for example, trimethylamine, triethylamine or pyridine.

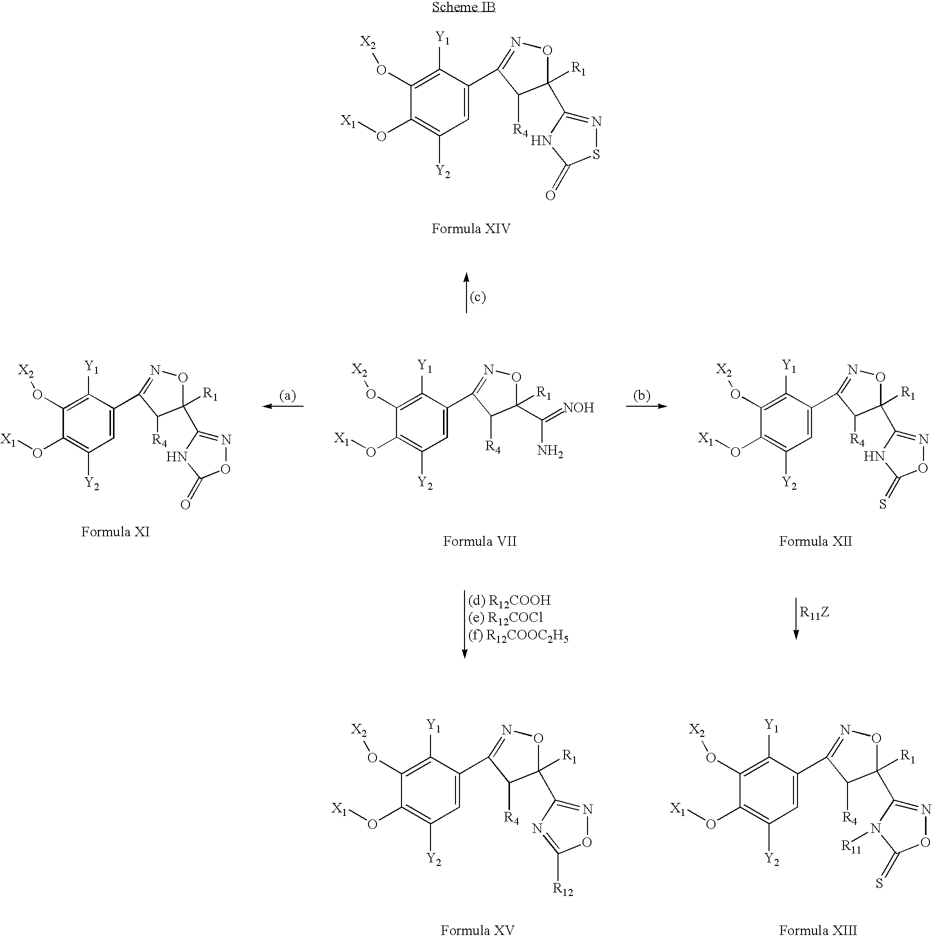

The compounds of Formulae XI-XV can be prepared according to scheme IB. Thus, reacting a compound of Formula VII (wherein X₁, X₂, Y₁, Y₂, R₁ and R₄ are the same as defined earlier) with
(a) methyl chloroformate to give a compound of Formula XI;
(b) thiocarbonyl diimidazole and 1,8-diazabicyclo[5.4.0]undec-7-one to give a compound of Formula XII, which on treatment with a compound of Formula R₁₁Z (wherein Z is halogen) gives a compound of Formula XIII (wherein R₁₁ is alkyl);
(c) thiocarbonyl diimidazole and boron trifluoride etherate to give a compound of Formula XIV;
(d) a compound of Formula R₁₂COOH,
(e) a compound of Formula R₁₂COCl or
(f) a compound of Formula R₁₂COOC₂H₅ to give a compound of Formula XV (wherein R₁₂ is alkyl, cycloalkyl, aryl, heteroaryl or heterocyclyl).

The reaction of a compound of Formula VII with methyl chloroformate to give a compound of Formula XI can be carried out in a solvent, for example, xylene, benzene or toluene.

The reaction of a compound of Formula VII with methyl chloroformate can be carried out in the presence of an organic base, for example, pyridine, trimethylamine or triethylamine.

The reaction of a compound of Formula VII with thiocarbonyl diimidazole and 1,8-diazabicyclo[5.4.0]undec-7-one to give a compound of Formula XII can be carried out in a solvent, for example, acetonitrile, acetone, dimethylformamide, dimethylsulfoxide or tetrahydrofuran.

The reaction of a compound of Formula XII with a compound of Formula R₁₁Z to give a compound of Formula XIII can be carried out in a solvent, for example, acetone, acetonitrile, tetrahydrofuran or dimethylformamide.

The reaction of a compound of Formula XII with a compound of Formula R₁₁Z can be carried out in the presence of an inorganic base, for example, sodium carbonate, sodium bicarbonate, potassium carbonate or potassium bicarbonate.

The reaction of a compound of Formula VII with a compound of Formula R₁₂COOH to give a compound of Formula XV can be carried out in the presence of isobutylchloroformate and an organic base, for example, triethylamine, dimethylamine or pyridine in a solvent, for example, dimethylformamide, tetrahydrofuran or acetonitrile.

The reaction of a compound of Formula VII to give a compound of Formula XV can be carried out in the presence of 1-ethyl-3-(3-dimethylaminopropyl)carbodiimide hydrochloride, 1-hydroxybenzotriazole and N-methylmorpholine.

The reaction of a compound of Formula VII with a compound of Formula R₁₂COCl to give a compound of Formula XV can be carried out in a solvent, for example, toluene, acetonitrile, acetone, dimethylformamide, dimethylsulphoxide or tetrahydrofuran.

The reaction of a compound of Formula VII with a compound of Formula R₁₂COOC₂H₅ to give a compound of Formula XV can be carried out in the presence of an inorganic base, for example, sodium carbonate, potassium carbonate or sodium hydride in a solvent, for example, dimethylformamide, tetrahydrofuran or acetonitrile.

The reaction of a compound of Formula VII with the compounds of Formula R₁₂COOH, R₁₂COCl and R₁₂COOC₂H₅ can be cared out in the presence of molecular sieves.

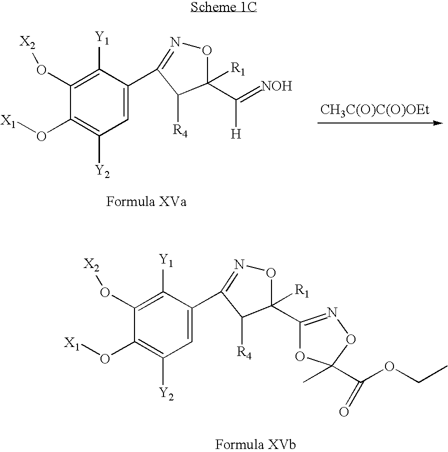

The compounds of Formula XVb can be prepared according to Scheme IC. Thus reacting a compound of Formula XVa with 2-oxo propionic acid ethyl ester gives a compound of Formula XVb (wherein X₁, X₂, Y₁, Y₂, R₁, and R₄ are the same as earlier). The reaction can be carried out in a solvent, for example, acetonitrile, acetone, dimethylformamide, dimethylsulphoxide or tetrahydrofuran.

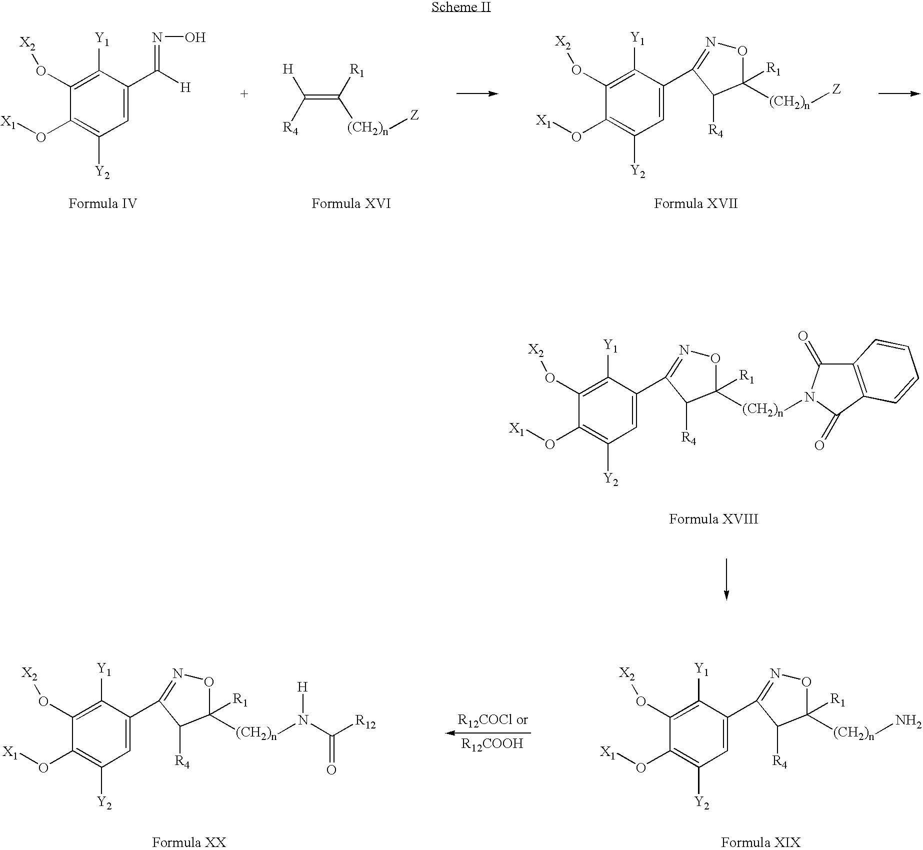

The compounds of Formula XX can be prepared according to Scheme II. Thus reacting a compound of Formula IV with a compound of Formula XVI to give a compound of Formula XVII (wherein X₁, X₂, Y₁, Y₂, R₁, R₄, Z and n are the same as defined earlier), which on treatment with potassium phthalamide gives a compound of Formula XVIII, which on treatment with a hydrazine hydrate gives a compound of Formula XIX, which is finally treated with a compound of Formula R₁₂COCl or R₁₂COOH to give a compound of Formula XX (wherein R₁₂ is the same as defined earlier).

The reaction of a compound of Formula IV with a compound of Formula XVI to give a compound of Formula XVII can be carried out in a solvent, for example, acetonitrile, acetone, dimethylformamide, dimethylsulphoxide or tetrahydrofuran.

The reaction of a compound of Formula XVII with potassium phthalamide to give a compound of Formula XVIII can be carried out in a solvent, for example, acetonitrile, acetone, dimethylformamide, dimethylsulphoxide or tetrahydrofuran.

The reaction of a compound of Formula XVIII with hydrazine hydrate to give a compound of Formula XIX can be carried out in a solvent, for example, methanol, ethanol, propanol, butanol, water or mixture thereof.

The reaction of a compound of Formula XIX with a compound of Formula R₁₂COCl to give a compound of Formula XX can be carried out in a solvent, for example, chloroform, dichloromethane or dichloroethane.

The reaction of a compound of Formula XIX with a compound of Formula R₁₂COCl can be carried out in the presence of an organic base, for example, trimethylamine, triethylamine or pyridine.

The reaction of a compound of Formula XIX with a compound of Formula R₁₂COOH to give a compound of Formula XX can be carried out in the presence of 1-ethyl-3-[3-(dimethylamino)propyl]carbodiimide, 1-hydroxybenzotriazole and N-methyl morpholine in a solvent, for example, dimethylformamide, dimethylsulfoxide or tetrahydrofuran.

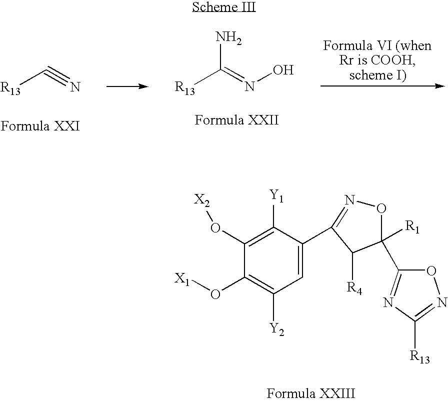

The compounds of Formula XXIII can be prepared according to Scheme III. Thus, reacting a compound of Formula XXI with hydroxylamine hydrochloride to give a compound of Formula XXII (wherein R₁₃ is alkyl, aryl or heteroaryl), which on reaction with a compound of Formula VI (when Rr is COOH, scheme I) gives a compound of Formula XXIII (wherein X₁, X₂, Y₁, Y₂, R₁ and R₄ are the same as defined earlier).

The reaction of a compound of Formula XXI to give a compound of Formula XXII can be carried out in the presence of sodium carbonate or potassium carbonate in a solvent, for example, methanol, ethanol propanol, n-butanol, water or mixture thereof.

The reaction of a compound of Formula XXII with a compound of Formula VI to give a compound of Formula XXIII can be carried out in a solvent, for example, dimethylformamide or dimethylsulfoxide.

The reaction of a compound of Formula XXII with a compound of Formula VI can be carried out in the presence of 1-hydroxybenzthiazole, N-methylmorpholine and a coupling agent, for example, 1-(3-dimethylaminopropyl)-3-ethyl carbodiimide hydrochloride or 1,3-dicyclohexyl carbodiimide.

The reaction of a compound of Formula XXII with a compound of Formula VI to give a compound of Formula XXIII can be carried out in the presence of sodium acetate or potassium acetate solvent, for example, methanol, ethanol, propanol, n-butanol, water or mixture thereof.

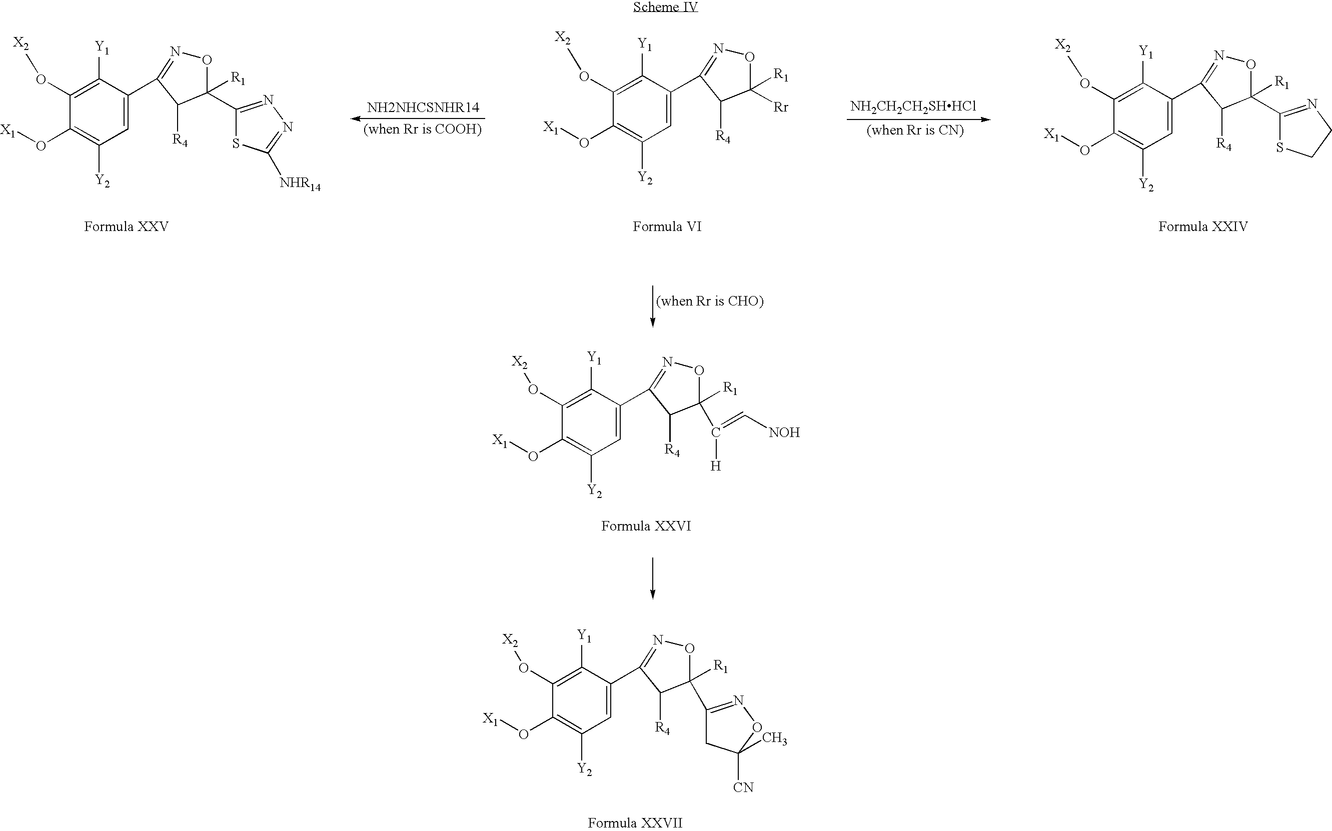

The compounds of Formula XXIV-XXVII can be prepared according to scheme IV. Thus, reacting a compound of
(1) Formula VI (when Rr is CN) with NH₂CH₂CH₂SH. HCl to give a compound of Formula XXIV (wherein X₁, X₂, Y₁, Y₂, R₁ and R₄ are the same as defined earlier);
(2) Formula VI (when Rr is COOH) with NH₂NHCSNHR₁₄ to give a compound of Formula XXV (wherein X₁, X₂, Y₁, Y₂, R₁, and R₄ are the same as defined earlier, R₁₄ represents hydrogen, alkyl or cycloalkyl); or
(3) Formula VI (when Rr is CHO) with hydroxylamine hydrochloride to give a compound of Formula XXVI which on reaction with methacrylonitrile gives a compound of Formula XXVII (wherein X₁, X₂, Y₁, Y₂, R₁ and R₄ are the same as defined earlier).

The reaction of a compound of Formula VI with NH₂CH₂CH₂SH. HCl to give a compound of Formula XXIV can be carried out in the presence of an organic base, for example, triethylamine, trimethylamine or pyridine in a solvent, for example, methanol, ethanol or isopropanol.

The reaction of a compound of Formula VI with NH₂NHCSNHR₁₄ to give a compound of Formula XXV can be carried out in the presence of POCl₃ in a solvent, for example, methanol or dioxane.

The reaction of a compound of Formula VI with hydroxylamine hydrochloride and sodium acetate to give a compound of Formula XXVI can be carried out in a solvent, for example, methanol, ethanol or isopropanol.

The reaction of a compound of Formula XXVI with methacrylonitrile to give a compound of Formula XXVII can be carried out in the presence of sodium hypochlorite in a solvent, for example, tetrahydrofuran, dimethylformamide, dimethylsulphoxide or acetonitrile.

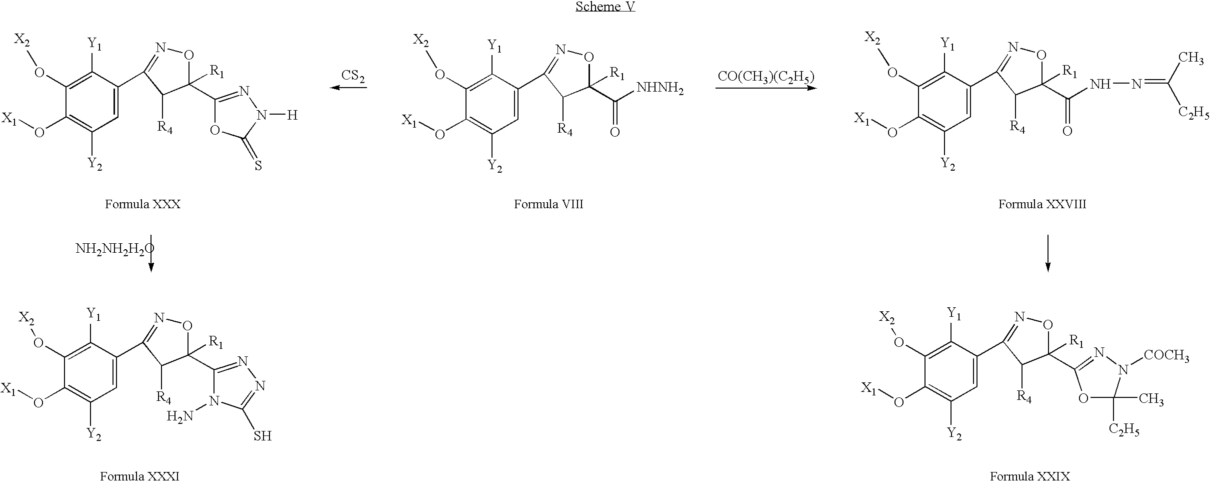

The compounds of Formula XXIX-XXXI can be prepared according to Scheme V. Thus, reacting a compound of Formula VIII
(1) with ethylmethylketone to give a compound of Formula XXVIII, which on treatment with acetic anhydride gives a compound of Formula XXIX (wherein X₁, X₂, Y₁, Y₂, R₁ and R₄ are the same as defined earlier)
(2) with carbon disulphide to give a compound of Formula XXX, which on treatment with hydrazine hydrate gives a compound of Formula XXXI (wherein X₁, X₂, Y₁, Y₂, R₁ and R₄ are the same as defined earlier).

The reaction of a compound of Formula VIII with ethylmethylketone to give a compound of Formula XXVIII can be carried out in a solvent, for example, methanol, ethanol or isopropanol.

The reaction of a compound of Formula XXVIII with acetic acid to give a compound of Formula XXIX can be carried out in the presence of an organic base, for example, pyridine, triethylamine or trimethylamine.

The reaction of a compound of Formula VIII with carbon disulphide to give a compound of Formula XXX can be carried out in the presence of an inorganic base, for example, sodium hydroxide, potassium hydroxide or calcium hydroxide.

The reaction of a compound of Formula VIII with carbon disulphide to give a compound of Formula XXX can be carried out in a solvent, for example, methanol, ethanol or isopropanol.

The reaction of a compound of Formula XXX with hydrazine hydrate to give a compound of Formula XXXI can be carried out in a solvent, for example, methanol, ethanol or isopropanol.

In the above schemes, where the specific solvents, bases, coupling agents etc., are mentioned, it is to be understood that other solvents, bases coupling agents etc., known to those skilled in the art may be used. Similarly, the reaction temperature and duration may be adjusted according to the desired needs.

An illustrative list of compounds of the invention are listed below (also shown in Table 1 2, 3, 4, 5, 6 and 7)
-3-[3-(3-Cyclopentyloxy-4-methoxyphenyl)-5-methyl-4,5-dihydroisoxazol-5-yl]-4H-[1,2,4]oxadiazol-5-one (Compound No. 1),
-3-[3-(3-Cyclopentyloxy-4-methoxyphenyl)-5-methyl-4,5-dihydroisoxazol-5-yl]-4H-[1,2,4]oxadiazole-5-thione (Compound No. 2),
-3-[3-(3-Cyclopentyloxy-4-methoxyphenyl)-5-methyl-4,5-dihydroisoxazol-5-yl]-4H-[1,2,4]thiadiazol-5-one (Compound No. 3),
-2-[3-(3-Cyclopentyloxy-4-methoxyphenyl)-5-methyl-4,5-dihydroisoxazol-5-yl]-[1,3,4]oxadiazole (Compound No. 4),
-2-[3-(3-Cyclopentyloxy-4-methoxyphenyl)-5-methyl-4,5-dihydroisoxazol-5-yl]-5-methyl-[1,3,4]oxadiazole (Compound No. 5),
-3-[3-(3-Cyclopentyloxy-4-methoxyphenyl)-5-methyl-4,5-dihydroisoxazol-5-yl]-4-methyl-4H-[1,2,4]oxadiazole-5-thione (Compound No. 6),
-3-{3-[3-(3-Cyclopentyloxy-4-methoxyphenyl)-5-methyl-4,5-dihydroisoxazol-5-yl]-[1,2,4]oxadiazol-5-yl}pyridine (Compound No. 7),
-5-tert-Butyl-3-[3-(3-Cyclopentyloxy-4-methoxyphenyl)-5-methyl-4,5-dihydroisoxazol-5-yl]-[1,2,4]oxadiazole (Compound No. 8),
-5-[3-(3-3-Cyclopentyloxy-4-methoxyphenyl)-5-methyl-4-,5-dihydroisoxazol-5-yl]-1H-tetrazole (Compound No. 9),
-3-(3-Cyclopentyloxy-4-methoxyphenyl)-5-methyl-4-,5-dihydroisoxazole-5-carbonitrile (Compound No. 10),
-Morpholine-4-carboxylic acid [3-(3-cyclopentyloxy-4-methoxyphenyl)-5-methyl-4,5-dihydroisoxazol-5-ylmethyl]amide (Compound No. 11),
—N-[3-(3-Cyclopentyloxy-4-methoxyphenyl)-5-methyl-4,5-dihydroisoxazol-5-ylmethyl]-4-fluoro-benzamide (Compound No. 12),
-Adamantane-1-carboxylic acid [3-(3-cyclopentyloxy-4-methoxyphenyl)-5-methyl-4,5-dihydroisoxazol-5-yl-methyl]amide (Compound No. 13),
-Furan-2-carboxylic acid [3-(3-cyclopentyloxy-4-methoxyphenyl)-5-methyl-4,5-dihydroisoxazol-5-yl-methyl]amide (Compound No. 14),
-2-(3-Cyclopentyloxy-4-methoxyphenyl)-N-[3-(3-cyclopentyloxy-4-methoxyphenyl)-5-methyl-4,5-dihydro-isoxazol-5-ylmethyl]-acetamide (Compound No. 15),
-1-[3-(3-Cyclopentyloxy-4-methoxyphenyl)-5-methyl-4,5-dihydro-isoxazol-5-yl-methyl]-3-(2-trifluoromethyl)-phenyl)-urea (Compound No. 16),
-1-[3-(3-Cyclopentyloxy-4-methoxyphenyl)-5-methyl-4,5-dihydro-isoxazol-5-yl-methyl]-3-(2,4-difluorophenyl)-urea (Compound No. 17),
-1-[3-(3-Cyclopentyloxy-4-methoxyphenyl)-5-methyl-4,5-dihydro-isoxazol-5-yl-methyl]-3-O-tolyl-urea (Compound No. 18),
-Morpholine-4-carboxylic acid [3-(3-cyclopentyloxy-4-methoxyphenyl)-5-methyl-4,5-dihydro-isoxazol-5-yl-methyl]-amide (Compound No. 19),
-3-(2-Chloro-6-trifluoromethylphenyl)-5-[3-(3-cyclopentyloxy-4-methoxyphenyl)-5-methyl-4,5-dihydro-isoxazol-5-yl-[1,2,4]oxadiazole (Compound No. 20),
-3-(2-Chloro-4-fluorophenyl)-5-[3-(3-cyclopentyloxy-4-methoxyphenyl)-5-methyl-4,5-dihydro-isoxazol-5-yl]-[1,2,4]oxadiazole (Compound No. 21),
-3-(4-Chloro-2-methoxyphenyl)-5-[3-(3-cyclopentyloxy-4-methoxyphenyl)-5-methyl-4,5-dihydro-isoxazol-5-yl]-[1,2,4]oxadiazole (Compound No. 22),
-3-(3-Chloro-4-fluorophenyl)-5-[3-(3-cyclopentyloxy-4-methoxyphenyl)-5-methyl-4,5-dihydro-isoxazol-5-yl]-[1,2,4]oxadiazole (Compound No. 23),
-3-(3-Chloro-4-methoxyphenyl)-5-[3-(3-cyclopentyloxy-4-methoxyphenyl)-5-methyl-4,5-dihydro-isoxazol-5-yl]-[1,2,4]oxadiazole (Compound No. 24),
-3-(3-Fluorophenyl)-5-[3-(3-cyclopentyloxy-4-methoxyphenyl)-5-methyl-4,5-dihydro-isoxazol-5-yl]-[1,2,4]oxadiazole (Compound No. 25),
-3-(3,4-Difluorophenyl)-5-[3-(3-cyclopentyloxy-4-methoxyphenyl)-5-methyl-4,5-dihydroisoxazol-5-yl]-[1,2,4]oxadiazole (Compound No. 26),
-3-(4-Methoxyphenyl)-5-[3-(3-cyclopentyloxy-4-methoxyphenyl)-5-methyl-4,5-dihydroisoxazol-5-yl]-[1,2,4]oxadiazole (Compound No. 27),
-3-(3,4-Dimethoxyphenyl)-5-[3-(3-cyclopentyloxy-4-methoxyphenyl)-5-methyl-4,5-dihydro-isoxazol-5-yl]-[1,2,4]oxadiazole (Compound No. 28),
-3-(2-Chloro-6-fluorophenyl)-5-[3-(3-cyclopentyloxy-4-methoxyphenyl)-5-methyl-4,5-dihydro-isoxazol-5-yl]-[1,2,4]oxadiazole (Compound No. 29),
-3-(2,5-Difluorophenyl)-5-[3-(3-cyclopentyloxy-4-methoxyphenyl)-5-methyl-4,5-dihydro-isoxazol-5-yl]-[1,2,4]oxadiazole (Compound No. 30),
-3-(2,6-Dichlorophenyl)-5-[3-(3-cyclopentyloxy-4-methoxyphenyl)-5-methyl-4,5-dihydro-isoxazol-5-yl]-[1,2,4]oxadiazole (Compound No. 31),
-3-(2,3-Dichlorophenyl)-5-[3-(3-cyclopentyloxy-4-methoxyphenyl)-5-methyl-4,5-dihydro-isoxazol-5-yl]-[1,2,4]oxadiazole (Compound No. 32),
-3-(2,4-Dichlorophenyl)-5-[3-(3-cyclopentyloxy-4-methoxyphenyl)-5-methyl-4,5-dihydro-isoxazol-5-yl]-[1,2,4]oxadiazole (Compound No. 33),
-3-(3,5-Dichlorophenyl)-5-[3-(3-cyclopentyloxy-4-methoxyphenyl)-5-methyl-4,5-dihydro-isoxazol-5-yl]-[1,2,4]oxadiazole (Compound No. 34),
-3-(2,5-Dichlorophenyl)-5-[3-(3-cyclopentyloxy-4-methoxyphenyl)-5-methyl-4,5-dihydro-isoxazol-5-yl]-[1,2,4]oxadiazole (Compound No. 35),
-3-(3,5-Difluorophenyl)-5-[3-(3-cyclopentyloxy-4-methoxyphenyl)-5-methyl-4,5-dihydro-isoxazol-5-yl]-[1,2,4]oxadiazole (Compound No. 36),
-3-(3-Chlorophenyl)-5-[3-(3-cyclopentyloxy-4-methoxyphenyl)-5-methyl-4,5-dihydro-isoxazol-5-yl]-[1,2,4]oxadiazole (Compound No. 37),
-3-(2,4-Difluoro-phenyl)-5-[3-(3-cyclopentyloxy-4-methoxyphenyl)-5-methyl-4,5-dihydro-isoxazol-5-yl]-[1,2,4]oxadiazole (Compound No. 38),
-3-(3,4-Dichlorophenyl)-5-[3-(3-cyclopentyloxy-4-methoxyphenyl)-5-methyl-4,5-dihydro-isoxazol-5-yl]-[1,2,4]oxadiazole (Compound No. 39),
-3-(4-Chlorophenyl)-5-[3-(3-cyclopentyloxy-4-methoxyphenyl)-5-methyl-4,5-dihydro-isoxazol-5-yl]-[1,2,4]oxadiazole (Compound No. 40),
-4-{5-[3-(3-Cyclopentyloxy-4-methoxyphenyl)-5-methyl-4,5-dihydro-isoxazol-5-yl]-[1,2,4]oxadiazole-3-yl}-phenylamine (Compound No. 41),
-3-Phenyl-5-[3-(3-cyclopentyloxy-4-methoxy-phenyl)-5-methyl-4,5-dihydro-isoxazol-5-yl]-[1,2,4]oxadiazole (Compound No. 42),
-3-(3,4-Dimethyl-phenyl)-5-[3-(3-cyclopentyloxy-4-methoxyphenyl)-5-methyl-4,5-dihydro-isoxazol-5-yl]-[1,2,4]oxadiazole (Compound No. 43),
-3-(2-Chlorophenyl)-5-[3-(3-cyclopentyloxy-4-methoxyphenyl)-5-methyl-4,5-dihydro-isoxazol-5-yl]-[1,2,4]oxadiazole (Compound No. 44),
-3-(4-Fluorophenyl)-5-[3-(3-cyclopentyloxy-4-methoxyphenyl)-5-methyl-4,5-dihydro-isoxazol-5-yl]-[1,2,4]oxadiazole (Compound No. 45),
-3-Methyl-5-[3-(3-cyclopentyloxy-4-methoxyphenyl)-5-methyl-4,5-dihydro-isoxazol-5-yl]-[1,2,4]oxadiazole (Compound No. 46),
-3-(3-Cyclopentyloxy-4-methoxyphenyl)-5-methyl-5-oxazol-5-yl-4,5-dihydro-isoxazole (Compound No. 47),
-5-[3-(3-Cyclopentyloxy-4-methoxyphenyl)-5-methyl-4,5-dihydro-isoxazol-5-yl]-[1,3,4]thiadiazol-2-yl-amine (Compound No. 48),
-{5-[3-(3-Cyclopentyloxy-4-methoxyphenyl)-5-methyl-4,5-dihydro-isoxazol-5-yl]-[1,3,4]thiadiazol-2-yl}-cyclopropylamine (Compound No. 49),
-1-{5-[3-(3-Cyclopentyloxy-4-methoxyphenyl)-5-methyl-4,5-dihydro-isoxazol-5-yl]-2-ethyl-2-methyl-[1,3,4]oxadiazol-3-yl}-ethanone (Compound No. 50),
-3-(3-Cyclopentyloxy-4-methoxyphenyl)-5-(4,5-dihydro-thiazol-2-yl)-5-methyl-4,5-dihydro-isoxazole (Compound No. 51),
-3′-(3-Cyclopentyloxy-4-methoxyphenyl)-5,5′-dimethyl-4,5,4′,5′-tetrahydro-[3,5′]biisoxazolyl-5-carbonitrile (Compound No. 52),
-3-{3-[3-(cyclopentyloxy)-4-methoxyphenyl]-5-methyl-4,5-dihydroisoxazol-5-yl}-5-methyl-1,4,2-dioxazole-5-carboxylic acid ethyl ester (Compound No. 53)
-4-Amino-5-[3-(3-cyclopentyloxy-4-methoxyphenyl)-5-methyl-4,5-dihydro-isoxazol-5-yl]-4H-[1,2,4]triazole-3-thiol (Compound No. 54),
-5-[3-(3-Cyclopentyloxy-4-methoxyphenyl)-5-methyl-4,5-dihydro-isoxazol-5-yl]-3H-[1,3,4]oxadiazole-2-thione (Compound No. 55),
-3-[3-(3-Cyclopentyloxy-4-methoxyphenyl)-5-methyl-4,5-dihydro-isoxazol-5-yl]-5-methyl-[1,2,4]oxadiazole (Compound No. 56),
-4-{3-[3-(3-Cyclopentyloxy-4-methoxyphenyl)-5-methyl-4,5-dihydro-isoxazol-5-yl]-[1,2,4]oxadiazol-5-yl}-pyridine (Compound No. 57),
-5-tert-Butyl-3-[3-(3-cyclopentyloxy-4-methoxyphenyl)-5-methyl-4,5-dihydro-isoxazol-5-yl]-[1,2,4]oxadiazole (Compound No. 58),
-3-[3-(3-Cyclopentyloxy-4-methoxyphenyl)-5-methyl-4,5-dihydro-isoxazol-5-yl]-5-(2-ethoxy-phenyl)-[1,2,4]oxadiazole (Compound No. 59),
-3-[3-(3-Cyclopentyloxy-4-methoxyphenyl)-5-methyl-4,5-dihydro-isoxazol-5-yl]-5-cyclopropyl-[1,2,4]oxadiazole (Compound No. 60),
-5-(3-Chlorophenyl)-3-[3-(3-cyclopentyloxy-4-methoxyphenyl)-5-methyl-4,5-dihydro-isoxazol-5-yl]-[1,2,4]oxadiazole (Compound No. 61),
-5-[3-(3-Cyclopentyloxy-4-methoxyphenyl)-5-methyl-4,5-dihydro-isoxazol-5-yl]-5-m-tolyl-[1,2,4]oxadiazole (Compound No. 62),
-3-[3-(3-Cyclopentyloxy-4-methoxyphenyl)-5-methyl-4,5-dihydro-isoxazol-5-yl]-5-(3,5-dimethyl-phenyl)-[1,2,4]oxadiazole (Compound No. 63),
-2,6-Dichloro-4-{3-[3-(3-cyclopentyloxy-4-methoxyphenyl)-5-methyl-4,5-dihydro-isoxazol-5-yl]-[1,2,4]oxadiazol-5-yl}-pyridine (Compound No. 64),
-3-[3-(3-Cyclopentyloxy-4-methoxyphenyl)-5-methyl-4,5-dihydro-isoxazol-5-yl]-5-isopropyl-[1,2,4]oxadiazole (Compound No. 65),
-5-Cyclohexyl-3-[3-(3-cyclopentyloxy-4-methoxyphenyl)-5-methyl-4,5-dihydro-isoxazol-5-yl]-[1,2,4]oxadiazole (Compound No. 66),
-3-[3-(3-Cyclopentyloxy-4-methoxyphenyl)-5-methyl-4,5-dihydro-isoxazol-5-yl]-5-fluoromethyl-[1,2,4]oxadiazole (Compound No. 67),
-3-[3-(3-Cyclopentyloxy-4-methoxyphenyl)-5-methyl-4,5-dihydro-isoxazol-5-yl]-5-(tetrahydro-furan-2-yl)-[1,2,4]oxadiazole (Compound No. 68),
-3-[3-(3-Cyclopentyloxy-4-methoxyphenyl)-5-methyl-4,5-dihydro-isoxazol-5-yl]-5-(4-fluoro-phenyl)-[1,2,4]oxadiazole (Compound No. 69),
-3-[3-(3-Cyclopentyloxy-4-methoxyphenyl)-5-methyl-4,5-dihydro-isoxazol-5-yl]-5-(3-fluorophenyl) [1,2,4]oxadiazol (Compound No. 70),
-{3-[3-(3-Cyclopentyloxy-4-methoxyphenyl)-5-methyl-4,5-dihydro-isoxazol-5-yl]-[1,2,4]oxadiazol-5-yl}-acetonitrile (Compound No. 71),
-4-{3-[3-(3-Cyclopentyloxy-4-methoxyphenyl)-5-methyl-4,5-dihydro-isoxazol-5-yl]-[1,2,4]oxadiazole-5-yl)-benzonitrile (Compound No. 72),
-3-[3-(3-Cyclopentyloxy-4-methoxyphenyl)-5-methyl-4,5-dihydro-isoxazol-5-yl]-5-trifluoromethyl-[1,2,4]oxadiazole (Compound No. 73),
-3-[3-(3-Cyclopentyloxy-4-methoxyphenyl)-5-methyl-4,5-dihydro-isoxazol-5-yl]-5-(3-methoxy-phenyl)-[1,2,4]oxadiazole (Compound No. 74),
-3-[3-(3-Cyclopentyloxy-4-methoxyphenyl-5-methyl-4,5-dihydro-isoxazol-5-yl]-5-(3,4-dimethoxy-phenyl)[1,2,4]oxadiazole (Compound No. 75),
-5-(2-Chlorophenyl)-3-[3-(3-cyclopentyloxy-4-methoxyphenyl)-5-methyl-4,5-dihydro-isoxazol-5-yl]-[1,2,4]oxadiazole (Compound No. 76),
-3-[3-(3-Cyclopentyloxy-4-methoxyphenyl)-5-methyl-4,5-dihydro-isoxazol-5-yl]-5-(2,4-dichloro-phenyl)-[1,2,4]oxadiazole (Compound No. 77),
-5-Cyclopentyl-3-[3-(3-cyclopentyloxy-4-methoxyphenyl)-5-methyl-4,5-dihydro-isoxazol-5-yl]-[1,2,4]oxadiazole (Compound No. 78),
-3-[3-(3-Cyclopentyloxy-4-methoxyphenyl)-5-methyl-4,5-dihydro-isoxazol-5-yl]-5-(3,4-dichloro-phenyl)-[1,2,4]oxadiazole (Compound No. 79),
-2-[3-(4-Difluoromethoxy-3-methoxyphenyl)-5-methyl-4,5-dihydro-isoxazol-5-yl]-[1,3,4]oxadiazole (Compound No. 80),
-2-{3-[3-(Bicyclo[2.2.1]hept-2-yloxy)-4-difluoromethoxyphenyl]-5-methyl-4,5-dihydro-isoxazol-5-yl}-[1,3,4]oxadiazole (Compound No. 81),
-2-{3-[3-benzyloxy-4-difluoromethoxyphenyl]-5-methyl-4,5-dihydro-isoxazol-5-yl}-[1,3,4]oxadiazole (Compound No. 82),
-2-[3-[4-Difluoromethoxy-3-ethoxyphenyl)-5-methyl-4,5-dihydro-isoxazol-5-yl]-[1,3,4]oxadiazole (Compound No. 83),
-2-[3-[4-Difluoromethoxy-3-isopropoxyphenyl)-5-methyl-4,5-dihydro-isoxazol-5-yl]-([1,3,4]oxadiazole (Compound No. 84),
-2-[3-(3-Cyclohexyloxy-4-difluoromethoxyphenyl)-5-methyl-4,5-dihydro-isoxazol-5-yl]-[1,3,4]oxadiazole (Compound No. 85),
-2-[3-(3-Butoxy-4-difluoromethoxyphenyl)-5-methyl-4,5-dihydro-isoxazol-5-yl]-[1,3,4]oxadiazole (Compound No. 86),
-2-[3-(3-Cycloheptyloxy-4-difluoromethoxyphenyl)-5-methyl-4,5-dihydro-isoxazol-5-yl]-[1,3,4]oxadiazole (Compound No. 87),
-2-[3-(4-Difluoromethoxy-3-propoxyphenyl)-5-methyl-4,5-dihydro-isoxazol-5-yl]-[1,3,4]oxadiazole (Compound No. 88),
-2-[3-(3,4-Bis-difluoromethoxyphenyl)-5-methyl-4,5-dihydro-isoxazol-5-yl]-[1,3,4]oxadiazole (Compound No. 89),
-2-[3-(3-Cyclopentyloxy-4-difluoromethoxyphenyl)-5-methyl-4,5-dihydro-isoxazol-5-yl]-[1,3,4]oxadiazole (Compound No. 90),
-2-[3-(3-Cyclopropylmethoxy-4-difluoromethoxyphenyl)-5-methyl-4,5-dihydro-isoxazol-5-yl]-[1,3,4]oxadiazole (Compound No. 91),
-2-[3-(3-Cyclopropylmethoxy-4-methoxy-phenyl)-5-methyl-4,5-dihydro-isoxazol-5-yl]-[1,3,4]oxadiazole (Compound No. 92),
-2-[3-(3-Difluoromethoxy-4-methoxyphenyl)-5-methyl-4,5-dihydro-isoxazol-5-yl]-[1,3,4]oxadiazole (Compound No. 93),
-3-(3-Cyclopropylmethoxy-4-difluoromethoxyphenyl)-5-methyl-4,5-dihydro-isoxazole-5-carbonitrile (Compound No. 94),
-3-(3-Cyclopentyloxy-4-difluoromethoxyphenyl)-5-methyl-4,5-dihydro-isoxazole-5-carbonitrile (Compound No. 95),
-3-(3,4-Bis-difluoromethoxyphenyl)-5-methyl-4,5-dihydro-isoxazole-5-carbonitrile (Compound No. 96),
-3-(3-Cyclopropylmethoxy-4-methoxyphenyl)-5-methyl-4,5-dihydro-isoxazole-5-carbonitrile (Compound No. 97),
-3-(3-Butoxy-4-difluoromethoxyphenyl)-5-methyl-4,5-dihydro-isoxazole-5-carbonitrile (Compound No. 98),
-3-(4-Difluoromethoxy-3-propoxyphenyl)-5-methyl-4,5-dihydro-isoxazole-5-carbonitrile (Compound No. 99),
-3-(4-Difluoromethoxy-3-isopropoxyphenyl)-5-methyl-4,5-dihydro-isoxazole-5-carbonitrile (Compound No. 100),
-3-[3-(Bicyclo[2.2.1]hept-2-yloxy)-4-difluoromethoxyphenyl]-5-methyl-4,5-dihydro-isoxazole-5-carbonitrile (Compound No. 101),
-3-(3-Benzyloxy-4-difluoromethoxyphenyl)-5-methyl-4,5-dihydro-isoxazole-5-carbonitrile (Compound No. 102),
-2-[3-(3-Cyclopentyloxy-4-methoxyphenyl)-5-methyl-4,5-dihydro-isoxazol-5-yl]-pyridine (Compound No. 103),
-3-[3-(Cyclopentyloxy-4-methoxyphenyl]-5-methyl-4,5-dihydroisoxazole-5-carboxylic acid hydrazide (Compound No. 104),

-Acetic acid (Z)-2-amino-2-{3-[3-(cyclopentyloxy)-4-methoxyphenyl]-5-methyl-4,5-dihydroisoxazol-5-yl}vinyl ester (Compound No. 105).

TABLE 1

| | |
|---|---|
| | Formula I |
| 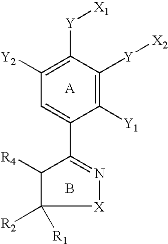 | |

(wherein R₄ = Y₁ = Y₂ = H, R₁ = X₁ = CH₃, X₂ = cyclopentyl, X = Y = O)

| Compound No. | R₂ |
|---|---|
| 1 | 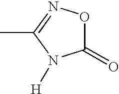 |
| 2 | 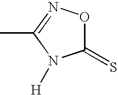 |
| 3 | 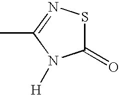 |
| 4 | 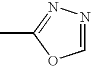 |
| 5 | 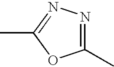 |
| 6 | 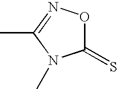 |
| 7 | 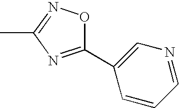 |
| 8 | 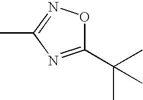 |
| 9 | 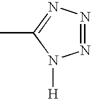 |
| 10 | CN |
| 104 | —CONHNH₂ |
| 105 | 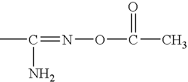 |

TABLE 2

| | |
|---|---|
| | Formula I |
| 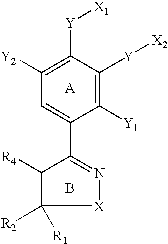 | |

(Formula I, wherein R₄ = Y₁ = Y₂ = H, R₁ = X₁ = CH₃, X₂ = cyclopentyl,
X = Y = O, R₂ = (CH₂)ₙ—NHCO—R₇, n = 1)

| Compound No. | R₇ |
|---|---|
| 11 | 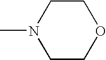 |
| 12 | 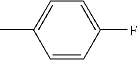 |
| 13 | 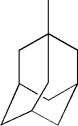 |
| 14 | 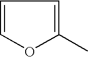 |
| 15 | 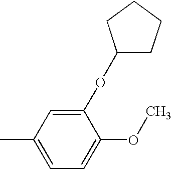 |

TABLE 3
(Formula I, wherein R₄ = Y₁ = Y₂ = H, R₁ = X₁ = CH₃, X₂ = cyclopentyl,
X = Y = O, R₂ = (CH₂)ₙ—NHCONHR₇, n = 1)

| Compound No. | R₇ |
|---|---|
| 16 | 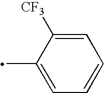 |
| 17 | 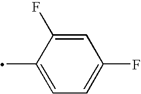 |
| 18 | 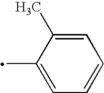 |
| 19 | 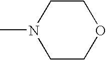 |

TABLE 4

| | |
|---|---|
| | Formula XXXII |
| 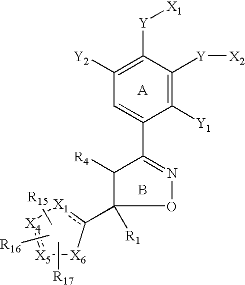 | |

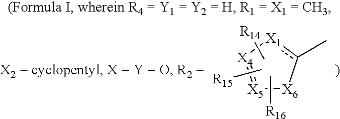

| Compound No. | R₂ |
|---|---|
| 20 | 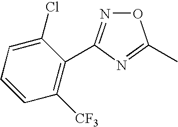 |
| 21 | 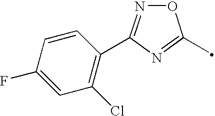 |
| 22 | 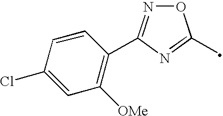 |
| 23 | 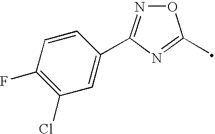 |
| 24 | 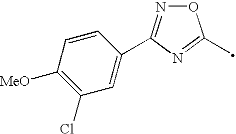 |
| 25 | 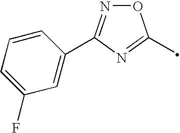 |
| 26 | 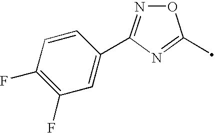 |
| 27 | 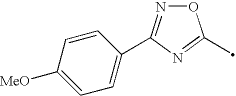 |
| 28 | 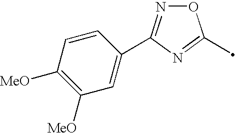 |
| 29 | 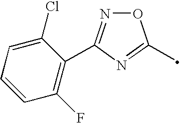 |
| 30 | 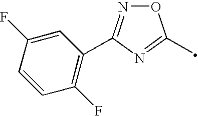 |
| 31 | 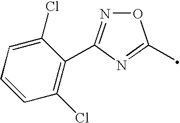 |
| 32 | 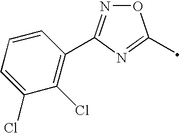 |
| 33 | 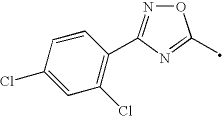 |
| 34 | 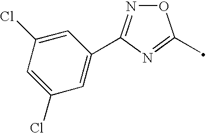 |
| 35 | 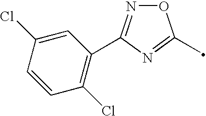 |
| 36 | 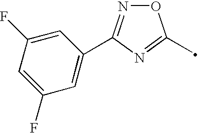 |
| 37 | 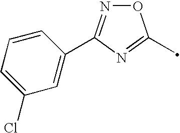 |
| 38 | 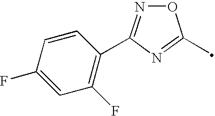 |
| 39 | 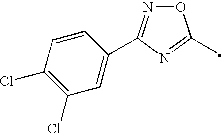 |
| 40 | 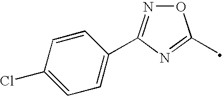 |
| 41 | 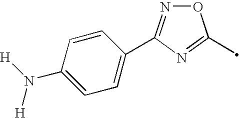 |
| 42 | 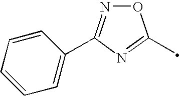 |
| 43 | 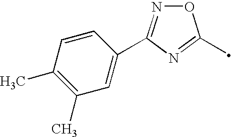 |
| 44 | 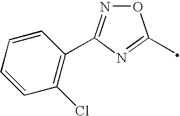 |
| 45 | 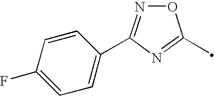 |
| 46 | 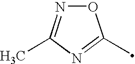 |
| 47 | 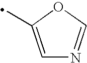 |
| 48 | 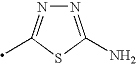 |
| 49 | 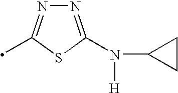 |
| 50 | 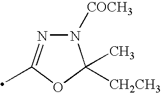 |
| 51 | 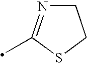 |
| 52 | 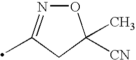 |
| 53 | 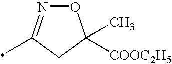 |
| 54 | 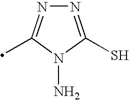 |
| 55 | 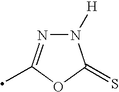 |

TABLE 5

| | |
|---|---|
| | Formula XXXIII |
| 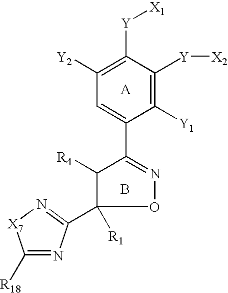 | |

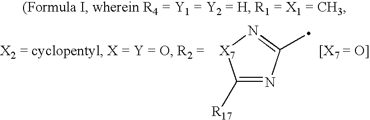

| Compound No. | R₂ |
|---|---|
| 56 | 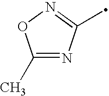 |
| 57 | 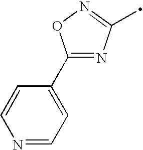 |
| 58 | 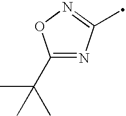 |
| 59 | 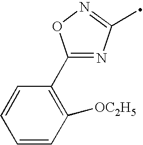 |
| 60 | 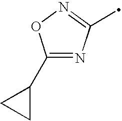 |
| 61 | 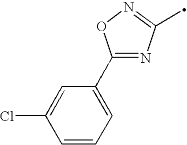 |
| 62 | 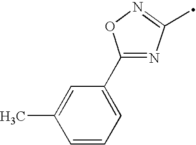 |
| 63 | 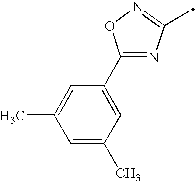 |
| 64 | 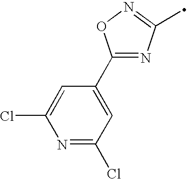 |
| 65 | 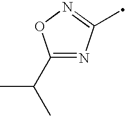 |
| 66 | 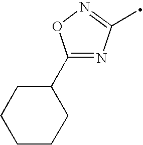 |
| 67 | 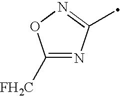 |
| 68 | 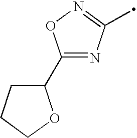 |
| 69 | 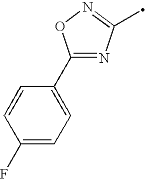 |
| 70 | 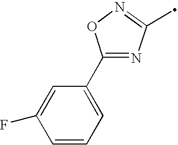 |
| 71 | 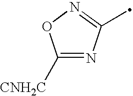 |
| 72 | 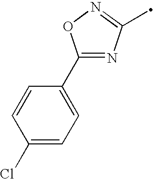 |
| 73 | 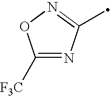 |
| 74 | 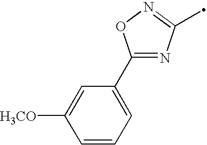 |
| 75 | 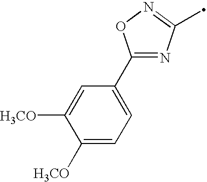 |
| 76 | 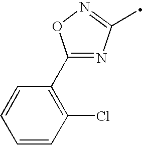 |
| 77 | 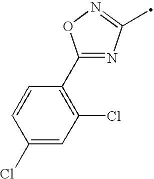 |
| 78 | 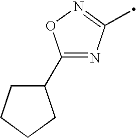 |
| 79 | 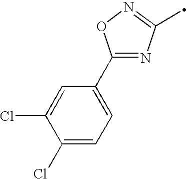 |

TABLE 6

| | |
|---|---|
| | Formula XXXII |
| 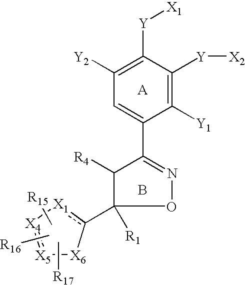 | |

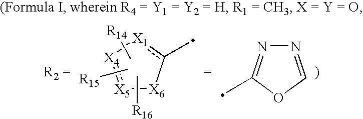

| Compound No. | X₂ | X₁ |
|---|---|---|
| 80 | —CH₃ | —CHF₂ |
| 81 | 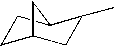 | —CHF₂ |
| 82 | 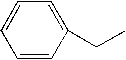 | —CHF₂ |
| 83 | 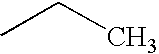 | —CHF₂ |
| 84 | 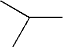 | —CHF₂ |
| 85 | 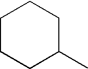 | —CHF₂ |
| 86 | CH₃—(CH₂)₃— | —CHF₂ |
| 87 | 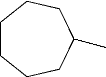 | —CHF₂ |
| 88 | 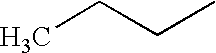 | —CHF₂ |
| 89 | CHF₂ | —CHF₂ |
| 90 | 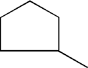 | —CHF₂ |
| 91 | 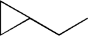 | —CHF₂ |
| 92 | —CHF₂ | —CH₃ |
| 93 | 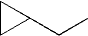 | —CH₃ |

TABLE 7

| | |
|---|---|
| | Formula XXXIV |
| 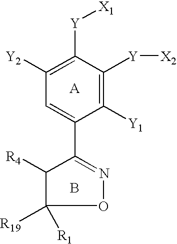 | |

(Formula I, wherein R₄ = Y₁ = Y₂ = H, R₁ = CH₃, X = Y = O)

| Compound No. | X₁ | X₂ | R₁₉ |
|---|---|---|---|
| 94 | —CHF₂ | 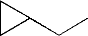 | CN |
| 95 | —CHF₂ | 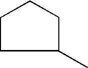 | CN |
| 96 | —CHF₂ | —CHF₂ | CN |
| 97 | —CH₃ | 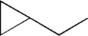 | CN |
| 98 | —CHF₂ | 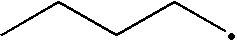 | CN |
| 99 | —CHF₂ | 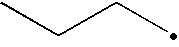 | CN |
| 100 | —CHF₂ | 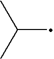 | CN |
| 101 | —CHF₂ | 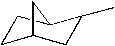 | CN |
| 102 | —CHF₂ | 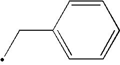 | CN |
| 103 | —CH₃ | 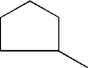 | 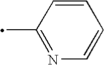 |

Examples set forth below demonstrate the synthetic procedures for the preparation of the representative compounds. The examples are provided to illustrate particular aspect of the disclosure and do not constrain the scope of the present invention as defined by the claims.

### EXPERIMENTAL DETAILS

### Example 1

### Preparation of 3-cyclopentyloxy-4-methoxybenzaldehyde

The title compound was prepared according to methods described in J. Med. Chem., (1994), 37, 1696-1703

### Example 2

### Preparation of 3-cyclopentyloxy-4-methoxybenzaldehyde oxime

To a stirred solution of 3-cyclopentyloxy-4-methoxybenzaldehyde (0.5 g, 2.2727 mmol, example 1) in ethanol (8 ml) was added hydroxylamine hydrochloride (0.473 g, 6.8181 mmol) and sodium acetate (0.56 g, 6.8181 mmol). The reaction mixture was allowed to stir at room temperature for 50 minutes. Ethanol was removed under reduced pressure and then residue was poured in water (20 ml) and organic compound was extracted with ethyl acetate (2×15 ml). Ethyl acetate layer was dried over anhydrous sodium sulphate, filtered and finally concentrated under reduced pressure to afford compound of Formula III.

¹H NMR (CDCl₃): 9.84 (s, 1H), 8.07 (s, 1H), 6.84-7.24 (m, 3H), 4.79-4.83 (m, 1H), 3.87 (s, 3H), 1.62-2.18 (m, 8H).

### Example 3

### Preparation of [3-(3-Cyclopentyloxy-4-methoxyphenyl)-5-methyl-4,5-dihydroisoxazole-5-carbonitrile (Compound No. 10)

3-cyclopentyloxy-4-methoxybenzaldehyde oxime (500 mg, 0.002 mole, example 2) was taken in 10 mL tetrahydrofuran. Methacrylonitrile (0.285 mL, 0.004 mole) was added and stirred. Sodium hypochlorite solution (10 mL, 20 times) was added dropwise. Reaction mixture was stirred vigorously at an ambient temperature. Tetrahydrofuran was removed under reduced pressure. Water was added and organic layer was extracted with ethyl acetate, dried and concentrated in vacuo. Residue was purified by column chromatography.

Yield: 63%; m.p.: 105°-106°; ¹H NMR: CDCl₃ δ=7.33-7.34 (d, 1H,), 6.96-6.99 (d, 1H,), 6.84-6.87 (d, 1H), 4.80-4.84 (m, 1H,), 3.86-3.88 (s, 3H), 3.80-3.86 (d, 1H), 3.36-3.41 (d, 1H), 1.80-2.0 (m, 8H), 1.56-1.63 (s, 3H); Mass (m/z) 301.5 (M⁺+1).

### Example 4

### Preparation of [3-(3-Cyclopentyloxy-4-methoxyphenyl)-N-hydroxy-5-methyl-4, 5-dihydroisoxazole-5-carboxamidine

[3-(3-Cyclopentyloxy-4-methoxyphenyl)-5-methyl-4,5-dihydroisoxazole-5-carbonitrile (200 mg, 0.0006 mole, example 3) was dissolved in 5 mL ethanol. To it anhydrous potassium carbonate (138 mg, 0.0009 mole) and hydroxylamine hydrochloride (92 mg, 0.0013 mole) was added & refluxed. Ethanol was removed under reduced pressure, water was added. Organic layer was extracted with ethyl acetate, washed with saturated sodium chloride solution, dried and concentrated in vacuo.

Yield: 95%; Mass (m/z): 334.21 (M⁺+1).

### Example 5

### Preparation of 3-(3-Cyclopentyloxy-4-methoxyphenyl)-5-methyl-4,5-dihydroisoxazole-5-carboxylic acid hydrazide (Compound No. 104)

To the ester (300 mg, 0.00086 mole, scheme I, Formula VI), hydrazine-hydrate (0.21 mL, 0.0043 mole) was added. Reaction mixture was heated at 120° C. Reaction mixture was cooled, water was added, solid, which was separated out, was filtered and dried under vacuum.

Yield: 49%; m.p: 159-160°; ¹H NMR (CDCl₃): δ 8.01 (s, 1H), 7.25-7.28 (d, 1H), 6.99-7.02 (d, 1H), 6.81-6.84 (d, 1H), 4.77-4.80 (m, 1H), 3.86 (s, 3H), 3.72-3.80 (d, 1H), 3.20-3.25 (d, 1H), 1.61-2.03 (m, 11H); Mass (m/z): 334.2 (M⁺+1).

### Example 6

### Preparation of 5-[3-(3-3-Cyclopentyloxy-4-methoxyphenyl)-5-methyl-4-,5-dihydroisoxazol-5-yl]-1H-tetrazole (Compound No. 9)

[3-(3-Cyclopentyloxy-4-methoxyphenyl)-5-methyl-4,5-dihydroisoxazole-5-carbonitrile (0.00029 mole, 100 mg, example 3), sodium azide (28 mg, 0.0004 mole) and triethylamine hydrochloride (0.0005 mole, 80 mg) was taken in 20 mL toluene. Reaction mixture was refluxed overnight. Toluene was removed and then added water to it. Extracted with ethyl acetate, washed with brine, dried and concentrated in vacuo.

Yield: 79%; m.p.: 161° C.; ¹H NMR (MeOD): δ 7.282-7.288 (d, 1H), 7.11-7.15 (d, 1H), 6.93-6.95 (d, 1H), 4.8 (m, 1H), 3.94-4.0 (d, 1H), 3.81 (s, 3H), 3.61-3.675 (d, 1H), 1.59-1.86 (m, 11H); Mass (m/z): 344.22 (M⁺+1).

### Example 7

### Preparation of 3-[3-(3-Cyclopentyloxy-4-methoxyphenyl)-5-methyl-4,5-dihydroisoxazol-5-yl]-4H-[1,2,4]oxadiazole-5-thione (Compound No. 2)

A mixture of [3-(3-Cyclopentyloxy-4-methoxyphenyl)-N-hydroxy-5-methyl-4,5-dihydroisoxazole-5-carboxamidine (0.0006 mole, 200 mg, example 4), thiocarbonyldiimidazole (0.0009 mole, 160 mg) and 1,8-diazabicyclo[5.4.0]undec-7-one (0.002 mol-358 mL) was taken in acetonitrile and stirred at an ambient temperature. Acetonitrile was removed under reduced pressure, water was added, organic layer was extracted with ethyl acetate, washed with saturated sodium chloride solution, dried and concentrated in vacuo. The residue was purified by column chromatography.

Yield: 50%; m.p: 172° C.; ¹H NMR (CDCl₃): δ 7.26 (d, 1H), 6.98-7.01 (d, 1H), 6.83-6.86 (d, 1H), 4.78-4.81 (m, 1H), 3.88-3.92 (d, 1H), 3.86 (s, 3H), 3.40-3.45 (d, 1H), 1.25-2.04 (m, 11H); Mass (m/z): 376.15 (M⁺+1).

### Example 8

### Preparation of 2-[3-(3-Cyclopentyloxy-4-methoxyphenyl)-5-methyl-4,5-dihydroisoxazol-5-yl]-[1,3,4]oxadiazole (Compound No. 4)

To 3-(3-Cyclopentyloxy-4-methoxyphenyl)-5-methyl-4,5-dihydroisoxazole-5-carboxylic acid hydrazide (250 mg, example 5) was added triethylorthoformate (5 mL). Reaction mixture was heated at 120° C. for 3 hours. Excess triethylorthoformate was evaporated and the residue was heated at 140° C. for 2 hours. Reaction mixture was diluted with water, saturated with potassium carbonate and extracted with ethyl acetate. Organic layer was dried, concentrated and purified by column chromatography.

Yield: 39%; m.p: 95° C.; ¹H NMR (CDCl₃): δ 8.44 (s, 1H), 7.37 (d, 1H), 7.05-7.08 (d, 1H), 6.85-6.88 (d, 1H), 4.82-4.83 (m, 1H), 4.19-4.24 (d, 1H), 3.88 (s, 3H), 3.43-3.49 (d, 1H), 1.62-2.30 (m, 8H), 1.24-1.28 (s, 3H); Mass (m/z): 344.16 (M⁺+1).

### Example 9

### Preparation of 2-[3-(3-Cyclopentyloxy-4-methoxyphenyl)-5-methyl-4,5-dihydro-isoxazol-5-yl]-5-methyl-[1,3,4]oxadiazole (Compound No. 5)

Prepared as described in example 8 by using triethylortho acetate instead of triethylortho formate.

Yield: 75%; m.p: oily; ¹H NMR (CDCl₃): δ 7.364 (s, 1H), 7.04-7.07 (d, 1H), 6.84-6.87 (d, 1H), 4.816 (s, 1H), 4.16-4.21 (d, 1H), 3.88 (s, 3H), 3.37-3.43 (d, 1H), 2.556 (s, 3H), 1.621-2.15 (m, 8H), 1.25-1.31 (m, 3H); Mass (m/z) 358.23 (M⁺+1).

### Example 10

### Preparation of 3-[3-(3-Cyclopentyloxy-4-methoxyphenyl)-5-methyl-4,5-dihydroisoxazol-5-yl]-4H-[1,2,4]thiadiazol-5-one (Compound No. 3)

Amidoxime (200 mg, 0.0006 mole, scheme L Formula VII) was taken in 3 mL tetrahydrofuran. To it thiocarbonyl diimidazole (160 mg, 0.0007 mole) was added. Reaction mixture was stirred at an ambient temperature. The reaction mixture was diluted with water, extracted with ethyl acetate, washed with water, dried and concentrated in vacuo. The residue was dissolved in tetrahydrofuran. Boron trifluoride etherate was added dropwise. The reaction mixture was stirred at an ambient temperature for 2 hours, diluted with water, extracted with ethyl acetate, dried, concentrated in vacuo and purified by column chromatography.

Yield: 23%; m.pt: 204° C.; ¹H NMR (CDCl₃): δ 7.319 (s, 1H), 7.015-7.042 (d, 1H), 6.83.6.85 (d, 1H), 4.80-4.82 (m, 1H), 3.95-4.00 (d, 1H), 3.87 (s, 3H), 3.33-3.39 (d, 1H), 1.83-2.04 (m, 8H), 1.25-1.62 (m, 3H); Mass (m/z): 376.14 (M⁺+1).

### Example 11

### Preparation of 3-[3-(3-Cyclopentyloxy-4-methoxyphenyl)-5-methyl-4,5-dihydro-isoxazol-5-yl]-4H-[1,2,4]oxadiazol-5-one (Compound No. 1)

Amidoxime (100 mg, 0.0003 mole, scheme I, Formula VII) was taken in dimethylformamide (1 mL). At 0° C. pyridine was added then at same temperature methyl chloroformate was added dropwise. The reaction mixture was stirred at 0° C. for about 30 minutes, water was added and organic layer was extracted with ethyl acetate, washed with saturated sodium chloride solution, dried and concentrated in vacuo. To residue xylene (5 ml) was added and refluxed for 18 hours. Xylene was removed under reduced pressure. The crude product was purified by column chromatography.

Yield: 37%; m.pt.: oily; ¹H NMR (CDCl₃): δ 7.29 (s, 1H), 7.01-7.04 (d, 1H), 6.84-6.87 (d, 1H), 4.78-4.81 (m, 2H), 3.91-3.96 (d, 1H), 3.88 (s, 3H), 3.31-3.40 (d, 1H), 1.22-2.00 (m, 11H); Mass (m/z): 360.18 (M⁺+1).

### Example 12

### Preparation of 3-{3-[3-(3-Cyclopentyloxy-4-methoxyphenyl)-5-methyl-4,5-dihydroisoxazol-5-yl]-[1,2,4]]oxadiazol-5-yl}-pyridine (Compound No. 7)

Nicotinic acid (0.0002 mole, 30 mg) was dissolved in dry dimethylformamide (1 mL). To it, molecular sieves (100 mg, 4 A°) and triethylamine (0.0003 mole, 0.05 mL) was added. The reaction mixture was cooled to −20° C. and isobutylchloroformate (0.0004 mole, 0.06 mL) was added. After 10 minutes amidoxime (0.0004 mole, 160 mg, scheme I, Formula VII) in dimethylformamide (2 mL) was added. The reaction mixture was stirred at an ambient temperature overnight. Some fresh molecular sieves were added. The reaction mixture was heated at 120° C. for 12 hours, mixture was filtered. To filtrate water was added, extracted with ethyl acetate, washed, dried and concentrated in vacuo. The residue was purified by column chromatography.

Yield: 25%; m.p.: oily; ¹H NMR (CDCl₃): δ 9.38 (s, 1H), 8.83-8.84 (d, 1H), 8.42-8.44 (d, 1H), 7.47-7.51 (m, 2H), 7.06-7.09 (d, 1H), 6.85-6.87 (d, 1H), 4.81-4.83 (m, 1H), 4.07-4.13 (d, 1H), 3.88 (s, 3H), 3.41-3.46 (d, 1H), 1.62-2.09 (m, 8H), 0.8-0.98 (m, 3H); Mass (m/z): 421.40 (M⁺+1).

The following compounds were prepared following the above procedure
-4-{3-[3-(3-Cyclopentyloxy-4-methoxyphenyl)-5-methyl-4,5-dihydro-isoxazol-5-yl]-[1,2,4]oxadiazol-5-yl}-pyridine (Compound No. 57),

Mass (m/z): 421.40 (M⁺+1)
-5-tert-Butyl-3-[3-(3-cyclopentyloxy-4-methoxyphenyl-5-methyl-4,5-dihydro-isoxazol-5-yl]-[1,2,4]oxadiazole (Compound No. 58),

Mass (m/z): 400.42 (M⁺+1)
-3-[3-(3-Cyclopentyloxy-4-methoxyphenyl)-5-methyl-4,5-dihydro-isoxazol-5-yl]-5-(2-ethoxy-phenyl)-[1,2,4]oxadiazole (Compound No. 59),

Mass (m/z): 464.43 (M⁺+1)

m.p.: 138.5-139° C.
-2,6-Dichloro-4-{3-[3-(3-cyclopentyloxy-methoxyphenyl)-5-methyl-4,5-dihydro-isoxazol-5-yl]-[1,2,4]oxadiazol-5-yl}-pyridine (Compound No. 64),

Mass (m/z): 489 (M⁺+1)

m.p.: 136.5° C.
-3-[3-(3-Cyclopentyloxy-4-methoxyphenyl)-5-methyl-4,5-dihydro-isoxazol-5-yl]-5-isopropyl-[1,2,4]oxadiazole (Compound No. 65),

m.p.: 87° C. Mass (m/z): 396.00 (M⁺+1)
-5-Cyclohexyl-3-[3-(3-cyclopentyloxy-4-methoxyphenyl)-5-methyl-4,5-dihydro-isoxazol-5-yl]-[1,2,4]oxadiazole (Compound No. 66),

Mass (m/z): 426.42 (M⁺+1)
-5-(2-Chlorophenyl)-3-[3-(3-cyclopentyloxy-4-methoxyphenyl)-5-methyl-4,5-dihydro-isoxazol-5-yl]-[1,2,4]oxadiazole (Compound No. 76),

Mass (m/z): 454.31 (M⁺+1)

m.p.: 122° C.
-3-[3-(3-Cyclopentyloxy-4-methoxyphenyl)-5-methyl-4,5-dihydro-isoxazol-5-yl]-5-(2,4-dichloro-phenyl)-[1,2,4]oxadiazole (Compound No. 77),

Mass (m/z): 488.25 (M⁺+1)

m.p.: 129° C.

### Example 13

### Preparation of 5-tert-Butyl-3-[3-(3-Cyclopentyloxy-4-methoxyphenyl)-5-methyl-4,5-dihydroisoxazol-5-yl]-[1.2.4]oxadiazole (Compound No. 8)

Amidoxime (100 mg, 0.0003 mole, scheme I, Formula VII) was taken in benzene (2 mL). Pivaloyl chloride (0.1 mL, 0.0009 mole) was added. The reaction mixture was refluxed for about 3 hours. Benzene was removed under reduced pressure. The residue was dissolved in ethyl acetate, washed with saturated sodium bicarbonate solution, dried and concentrated in vacuo. The residue was taken is dimethylformamide (5 mL) and refluxed for 3 hours. Dimethylformamide was removed under reduced pressure, water was added, extracted with ethyl acetate, dried and concentrated in vacuo.

Yield: 25%; m.p.: sticky solid; ¹H NMR (CDCl₃): δ 7.39-7.40 (d, 1H), 7.04-7.08 (d, 1H), 6.84-6.87 (d, 1H), 4.80-4.83 (m, 1H), 4.02-4.07 (d, 1H), 3.87 (s, 3H), 3.31-3.36 (d, 1H), 1.74-1.96 (m, 8H), 1.43 (s, 9H), 1.24-1.35 (m, 3H); Mass (m/z): 400.42 (M⁺+1).

### Example 14

### Preparation of 3-[3-(3-Cyclopentyloxy-4-methoxyphenyl)-5-methyl-4,5-dihydroisoxazol-5-yl]-4-methyl-4H-[1,2,4]oxadiazole-5-thione (Compound No. 6)

3-[3-(3-Cyclopentyloxy-4-methoxyphenyl)-5-methyl-4,5-dihydroisoxazol-5-yl]-4H-[1,2,4]oxadiazole-5-thione (0.0001 mole, 40 mg, example 7), was dissolved in acetone (2 mL). To it potassium carbonate (0.001 mole, 147 mg) and methyliodide (0.0002 mole, 0.016 mL) were added. The reaction mixture was refluxed for overnight. Filtered to remove potassium carbonate, washed with acetone. From filtrate, acetone was removed under reduced pressure to give a low melting solid compound.

Yield: 72%; ¹H NMR (CDCl₃): δ 7.38 (d, 1H), 7.03-7.06 (d, 1H), 6.83-6.86 (d, 1H), 4.80-4.82 (m, 1H), 3.97-4.02 (d, 1H), 3.87 (s, 3H), 3.31-3.37 (d, 1H), 2.72 (s, 3H), 1.80-1.99 (m, 8H), 1.26-1.32 (m, 3M); Mass (m/z): 390.38 (M⁺+1).

### Example 15

### General Method of Preparation of Compound of Formula XX (wherein R₁═X₁═CH₃, Y₁═R₄═Y₂═H, X₂=cyclopentyl and n=1)

Method A: To a stirred solution of 3-(3-Cyclopentyloxy-4-methoxy-phenyl)-5-methyl-4,5-dihydro-isoxazol-5-yl]-methylamine (0.3569 mmol, 1 equiv, Scheme II, Formula XIX) in 2 mL chloroform was added triethylamine (2.6767 mmol, 7.5 equiv). Compound of Formula R₁₂COCl (0.3925 mmol, 1.1 equiv) was added dropwise over a period of 15 minutes with stirring the solution vigorously. The reaction was allowed to stir at an ambient temperature. The reaction mixture was quenched by adding 5 mL water. The resulting mixture was extracted with chloroform. The organic layer was thoroughly washed with water and was dried over anhydrous sodium sulphate, filtered and concentrated over buchi to afford the crude product. The crude product was purified over silica gel column (100-200 mesh) using hexane and ethyl acetate mixture as eluent.

The following compounds were prepared following the above general procedure

-Morpholine-4-carboxylic acid [3-(3-cyclopentyloxy-4-methoxy-phenyl)-5-methyl-4,5-dihydro-isoxazol-5-ylmethyl]-amine (Compound No. 11),

Mass (m/z): 418.30 (M⁺+1),
—N-[3-(3-Cyclopentyloxy-4-methoxy-phenyl)-5-methyl-4,5-dihydro-isoxazol-5-ylmethyl]-4-fluoro-benzamide (Compound No. 12),

Mass (m/z): 427.27 (M⁺+1),
-Adamantane-1-carboxylic acid [3-(3-cyclopentyloxy-4-methoxy-phenyl)-5-methyl-4,5-dihydro-isoxazol-5-yl-methyl]amide (Compound No. 13),

Mass (m/z): 467.44 (M⁺+1),
-Furan-2-carboxylic acid [3-(3-cyclopentyloxy-4-methoxy-phenyl)-5-methyl-4,5-dihydro-isoxazol-5-ylmethyl]-amide (Compound No. 14),

Mass (m/z): 339.24 (M⁺+1),
-1-[3-(3-Cyclopentyloxy-4-methoxyphenyl)-5-methyl-4,5-dihydro-isoxazol-5-yl-methyl]-3-(2-trifluoromethyl)-phenyl)-urea (Compound No. 16),

Mass (m/z): 492.40 (M⁺+1)
-1-[3-(3-Cyclopentyloxy-4-methoxyphenyl)-5-methyl-4,5-dihydro-isoxazol-5-yl-methyl]-3-(2,4-difluorophenyl)-urea (Compound No. 17),

Mass (m/z): 460.31 (M⁺+1)
-1-[3-(3-Cyclopentyloxy-4-methoxyphenyl)-5-methyl-4,5-dihydro-isoxazol-5-yl-methyl]-3-O-tolyl-urea (Compound No. 18),

Mass (m/z): 438.31 (M⁺+1)
-Morpholine-4-carboxylic acid [3-(3-cyclopentyloxy-4-methoxyphenyl)-5-methyl-4,5-dihydro-isoxazol-5-yl-methyl]-amide (Compound No. 19),

Mass (m/z): 418.30 (M⁺+1)

Method B: To a solution of 3-(3-Cyclopentyloxy-4-methoxy-phenyl)-5-methyl-4,5-dihydro-isoxazol-5-yl]-methylamine (0.3407 mmol, 1 equiv, scheme II, Formula XX and R₁₂COOH (0.3407 mmol, 1 equiv.) in 0.8 mL dry dimethylformamide at 0° C. was added 1-hydroxybenzotriazole (0.3407 mmol, 1 equiv) and N-methylmorpholine (1.3628 mmol, 4 equiv.). The reaction mixture was allowed to stir at 0° C. for 30 minutes. Thereafter, 1-[3-(dimethylamino)propyl-3-ethyl]carbodiimide hydrochloride (0.6814 mmol, 2 equiv.) was added to the reaction mixture and reaction was continued at 0° C. for 1 hour and thereafter at an ambient temperature for 20 hours. The reaction was quenched by adding water. The resulting reaction mixture was extracted with ethyl acetate. The ethyl acetate layer was dried over anhydrous sodium sulphate, concentrated in vacuo to afford the crude product. The crude product was purified over silica gel column (100-200 mesh) using hexane and ethyl acetate mixture as eluent.

The following compound was prepared following the above procedure (Method B)

-2-(3-Cyclopentyloxy-4-methoxy-phenyl)-N-[3-(3-cyclopentyloxy-4-methoxy-phenyl)-5-methyl-4,5-dihydro-isoxazol-5-ylmethyl]-acetamide (Compound No. 15)

Mass (m/z): 523.37 (M⁺+1)

### Example 16

### General Method of Preparation of Compound of Formula IX (wherein R₄═Y₁═Y₂═H, R₁═CH₃, X₁═CHF₂, X═Y═O, R₁₁═H)

Step 1: Preparation of Compound of Formula VI

Oxime (Formula IV, scheme I, 1 equiv.) and methyl methacrylate (10 equiv.) were taken in tetrahydrofuran. At ambient temperature sodium hypochlorite solution was added dropwise. The reaction mixture was stirred at room temperature for overnight. Tetrahydrofuran was removed under reduced pressure. Water was added and extracted with ethyl acetate. The mixture washed with saturated sodium chloride solution, dried over anhydrous sodium sulfate and concentrated in vacuo to give pure compound.

Step 2: Preparation of Compound of Formula VIII

To the ester compound (Formula VI, step 1) hydrazine hydrate (10 equiv.) was added and allowed to stir at 120° C. for about 3 hours. When the reaction was complete, it was cooled and water was added. Solids which separated out were filtered, dissolved in ethyl acetate, washed with water, dried over anhydrous sodium sulfate and concentrated in vacuo to give pure compound.

Step 3: Preparation of Compound of Formula IX

Hydrazide (Formula VIII, step 2) and triethylorthoformate (5 ml per mmole) were heated at 120° C. for about 3 hours. Excess triethylorthoformate was evaporated and the residue heated for further about 2 hours at 140° C. When the reaction was complete, the reaction mixture was diluted with water, saturated with potassium carbonate and extracted with ethyl acetate. Organic layer was dried and concentrated in vacuo. Purification was done by column chromatography to give pure compound.

The following compounds were prepared following the above general procedure

-2-[3-(4-Difluoromethoxy-3-methoxyphenyl)-5-methyl-4,5-dihydro-isoxazol-5-yl]-[1,3,4]oxadiazole (Compound No. 80)

Mass (m/z): 326.12 (M⁺+1),
-2-{3-[3-(Bicyclo[2.2.1]hept-2-yloxy)-4-difluoromethoxyphenyl]-5-methyl-4,5-dihydro-isoxazol-5-yl}-[1,3,4]oxadiazole (Compound No. 81),

Mass (m/z): (M⁺+1)
-2-{3-[3-(benzyloxy)-4-difluoromethoxyphenyl]-5-methyl-4,5-dihydro-isoxazol-5-yl}-[1,3,4]oxadiazole (Compound No. 82)

Mass (m/z): 402.11 (M⁺+1),
-2-[3-[4-Difluoromethoxy-3-ethoxyphenyl)-5-methyl-4,5-dihydro-isoxazol-5-yl]-[1,3,4]oxadiazole (Compound No. 83)

Mass (m/z): 340.12 (M⁺+1),
-2-[3-[4-Difluoromethoxy-3-isopropoxyphenyl)-5-methyl-4,5-dihydro-isoxazol-5-yl]-[1,3,4]oxadiazole (Compound No. 84)

Mass (m/z): 354.0 (M⁺+1),
-2-[3-(3-Cyclohexyloxy-4-difluoromethoxyphenyl)-5-methyl-4,5-dihydro-isoxazol-5-yl]-[1,3,4]oxadiazole (Compound No. 85)

Mass (m/z): 394.16 (M⁺+1),
-2-[3-(3-Butoxy-4-difluoromethoxyphenyl)-5-methyl-4,5-dihydro-isoxazol-5-yl]-[1,3,4]oxadiazole (Compound No. 86)

Mass (m/z): 368.09 (M⁺+1),
-2-[3-(3-Cycloheptyloxy-4-difluoromethoxyphenyl)-5-methyl-4,5-dihydro-isoxazol-5-yl]-[1,3,4]oxadiazole (Compound No. 87)

Mass (m/z): 408.17 (M⁺+1),
-2-[3-(4-Difluoromethoxy-3-propoxyphenyl)-5-methyl-4,5-dihydro-isoxazol-5-yl]-[1,3,4]oxadiazole (Compound No. 88)

Mass (m/z): 354.14 (M⁺+1),
-2-[3-(3,4-Bis-difluoromethoxyphenyl)-5-methyl-4,5-dihydro-isoxazol-5-yl]-[1,3,4]oxadiazole (Compound No. 89)

Mass (m/z): 362.21 (M⁺+1),
-2-[3-(3-Cyclopentyloxy-4-difluoromethoxyphenyl)-5-methyl-4,5-dihydro-isoxazol-5-yl]-[1,3,4]oxadiazole (Compound No. 90)

Mass (m/z): 380.19 (M⁺+1),
-2-[3-(3-Cyclopropylmethoxy-4-difluoromethoxyphenyl)-5-methyl-4,5-dihydro-isoxazol-5-yl]-[1,3,4]oxadiazole (Compound No. 91)

Mass (m/z): 366.18 (M⁺+1),
-2-[3-(3-Cyclopropylmethoxy-4-methoxy-phenyl)-5-methyl-4,5-dihydro-isoxazol-5-yl]-[1,3,4]oxadiazole (Compound No. 92),

Mass (m/z): 330.18 (M⁺+1)
-2-[3-(3-Difluoromethoxy-4-methoxyphenyl)-5-methyl-4,5-dihydro-isoxazol-5-yl]-[1,3,4]oxadiazole (Compound No. 93)

Mass (m/z): 326.11 (M⁺+1),

### Example 17

### General Method of Preparation of Compound of Formula XXIV (wherein R₄═Y₁═Y₂═H, R₁═X₁═CH₃, X₂=cyclopentyl, X═Y═O)

Step 1: Preparation of Compound of Formula XXIII

Nitriles (Formula XXII, Scheme III, 1 equiv.) was taken in solution of ethanol/water (1:4) and stirred for about 5 minutes. To this hydroxylamine hydrochloride (3.7 equiv.) and sodium carbonate (1.8 equiv.) were added and stirred for about 10 minutes at ambient temperature. The reaction mixture was stirred at reflux for about 18 hours. Ethanol was removed under reduced pressure. Water was added and triturated. Solid which precipitates out was filtered and dried under vacuo to give the desired amidoxime.

Step 2: Preparation of Compound of Formula XXIV

Acid (Formula VI, Scheme I, 1 equiv.) and amidoxime (Formula XXII, step 1, 1.1 equiv.) was taken in dry dimethylformamide. At 0° C. hydroxybenzotriazole (1 equiv.) and N-methyl morpholine (4 equiv.) were added and stirred at 0° C. for about one hour. At the same temperature 1-(3-dimethylaminopropyl)-3-ethyl carbodiimide hydrochloride (2 equiv.) was added. The reaction mixture was stirred at room temperature for about 24 hours. Water was added, extracted with ethyl acetate, dried and concentrated in vacuo. Solid, which formed, was taken in ethanol:water (7:1) and sodium acetate (1.5 equiv.) was added. Reaction mixture was refluxed at 80-90° C. for about 3 hours. Cooled, solid, which separated out, was filtered and recrystallized with ethanol.

The following compounds were prepared following the above general procedure

-3-(2-Chloro-6-trifluoromethylphenyl)-5-[3-(3-cyclopentyloxy-4-methoxyphenyl)-5-methyl-4,5-dihydro-isoxazol-5-yl-[1,2,4]oxadiazole (Compound No. 20)

Mass (m/z): 522.22 (M⁺+1),
-3-(2-Chloro-4-fluorophenyl)-5-[3-(3-cyclopentyloxy-4-methoxyphenyl)-5-methyl-4,5-dihydro-isoxazol-5-yl]-[1,2,4]oxadiazole (Compound No. 21)

Mass (m/z): 472.22 (M⁺+1),
-3-(4-Chloro-2-methoxyphenyl)-5-[3-(3-cyclopentyloxy-4-methoxyphenyl)-5-methyl-4,5-dihydro-isoxazol-5-yl]-[1,2,4]oxadiazole (Compound No. 22)

Mass (m/z): 484.25 (M⁺+1),
-3-(3-Chloro-4-fluorophenyl)-5-[3-(3-cyclopentyloxy-4-methoxyphenyl)-5-methyl-4,5-dihydro-isoxazol-5-yl]-[1,2,4]oxadiazole (Compound No. 23)

Mass (m/z): 472.22 (M⁺1),
-3-(3-Chloro-4-methoxyphenyl)-5-[3-(3-cyclopentyloxy-4-methoxyphenyl)-5-methyl-4,5-dihydro-isoxazol-5-yl]-[1,2,4]oxadiazole (Compound No. 24)

Mass (m/z): 484.25 (M⁺+1),
-3-(3-Fluorophenyl)-5-[3-(3-cyclopentyloxy-4-methoxyphenyl)-5-methyl-4,5-dihydro-isoxazol-5-yl]-[1,2,4]oxadiazole (Compound No. 25)

Mass (m/z): 438.21 (M⁺+1),
-3-(3,4-Difluorophenyl)-5-[3-(3-cyclopentyloxy-4-methoxyphenyl)-5-methyl-4,5-dihydroisoxazol-5-yl]-[1,2,4]oxadiazole (Compound No. 26)

Mass (m/z): 456.2 (M⁺+1),
-3-(4-Methoxyphenyl)-5-[3-(3-cyclopentyloxy-4-methoxyphenyl)-5-methyl-4,5-dihydroisoxazol-5-yl]-[1,2,4]oxadiazole (Compound No. 27)

Mass (m/z): 450.27 (M⁺+1),
-3-(3,4-Dimethoxyphenyl)-5-[3-(3-cyclopentyloxy-4-methoxyphenyl)-5-methyl-4,5-dihydro-isoxazol-5-yl]-[1,2,4]oxadiazole (Compound No. 28)

Mass (m/z): 480.27 (M⁺+1),
-3-(2-Chloro-6-fluorophenyl)-5-[3-(3-cyclopentyloxy-4-methoxyphenyl)-5-methyl-4,5-dihydro-isoxazol-5-yl]-[1,2,4]oxadiazole (Compound No. 29)

Mass (m/z): 472.22 (M⁺+1),
-3-(2,5-Difluoro-phenyl)-5-[3-(3-cyclopentyloxy-4-methoxyphenyl)-5-methyl-4,5-dihydro-isoxazol-5-yl]-[1,2,4]oxadiazole (Compound No. 30)

Mass (m/z): 456.22 (M⁺+1),
-3-(2,6-Dichlorophenyl)-5-[3-(3-cyclopentyloxy-4-methoxyphenyl)-5-methyl-4,5-dihydro-isoxazol-5-yl]-[1,2,4]oxadiazole (Compound No. 31)

Mass (m/z): 488.18 (M⁺+1),
-3-(2,3-Dichloro-phenyl)-5-[3-(3-cyclopentyloxy-4-methoxyphenyl)-5-methyl-4,5-dihydro-isoxazol-5-yl]-[1,2,4]oxadiazole (Compound No. 32)

Mass (m/z): 488.18 (M⁺+1),
-3-(2,4-Dichlorophenyl)-5-[3-(3-cyclopentyloxy-4-methoxyphenyl)-5-methyl-4,5-dihydro-isoxazol-5-yl]-[1,2,4]oxadiazole (Compound No. 33)

Mass (m/z): 488.18 (M⁺+1),
-3-(3,5-Dichlorophenyl)-5-[3-(3-cyclopentyloxy-4-methoxyphenyl)-5-methyl-4,5-dihydro-isoxazol-5-yl]-[1,2,4]oxadiazole (Compound No. 34)

Mass (m/z): 488.18 (M⁺+1),
-3-(2,5-Dichlorophenyl)-5-[3-(3-cyclopentyloxy-4-methoxyphenyl)-5-methyl-4,5-dihydro-isoxazol-5-yl]-[1,2,4]oxadiazole (Compound No. 35)

Mass (m/z): 488.18 (M⁺+1),
-3-(3,5-Difluorophenyl)-5-[3-(3-cyclopentyloxy-4-methoxyphenyl)-5-methyl-4,5-dihydro-isoxazol-5-yl]-[1,2,4]oxadiazole (Compound No. 36)

Mass (m/z): 456.28 (M⁺+1),
-3-(3-Chlorophenyl)-5-[3-(3-cyclopentyloxy-4-methoxyphenyl)-5-methyl-4,5-dihydro-isoxazol-5-yl]-[1,2,4]oxadiazole (Compound No. 37)

Mass (m/z): 454.20 (M⁺+1),
-3-(2,4-Difluorophenyl)-5-[3-(3-cyclopentyloxy-4-methoxyphenyl)-5-methyl-4,5-dihydro-isoxazol-5-yl]-[1,2,4]oxadiazole (Compound No. 38)

Mass (m/z): 456.27 (M⁺+1),
-3-(3,4-Dichlorophenyl)-5-[3-(3-cyclopentyloxy-4-methoxyphenyl)-5-methyl-4,5-dihydro-isoxazol-5-yl]-[1,2,4]oxadiazole (Compound No. 39)

Mass (m/z): 488.21 (M⁺+1),
-3-(4-Chlorophenyl)-5-[3-(3-cyclopentyloxy-4-methoxyphenyl)-5-methyl-4,5-dihydro-isoxazol-5-yl]-[1,2,4]oxadiazole (Compound No. 40)

Mass (m/z): 434.24 (M⁺+1),
-4-{5-[3-(3-Cyclopentyloxy-4-methoxyphenyl)-5-methyl-4,5-dihydro-isoxazol-5-yl]-[1,2,4]oxadiazole-3-yl}-phenylamine (Compound No. 41)

Mass (m/z): 435.27 (M⁺+1),
-3-Phenyl-5-[3-(3-cyclopentyloxy-4-methoxyphenyl)-5-methyl-4,5-dihydro-isoxazol-5-yl]-[1,2,4]oxadiazole (Compound No. 42)

Mass (m/z): 420.30 (M⁺+1),
-3-(3,4-Dimethylphenyl)-5-[3-(3-cyclopentyloxy-4-methoxyphenyl)-5-methyl-4,5-dihydro-isoxazol-5-yl]-[1,2,4]oxadiazole (Compound No. 43)

Mass (m/z): 448.32 (M⁺+1),
-3-(2-Chlorophenyl)-5-[3-(3-cyclopentyloxy-4-methoxyphenyl)-5-methyl-4,5-dihydro-isoxazol-5-yl]-[1,2,4]oxadiazole (Compound No. 44),

Mass (m/z): 453.5 (M⁺+1),
-3-(4-Fluorophenyl)-5-[3-(3-cyclopentyloxy-4-methoxyphenyl)-5-methyl-4,5-dihydro-isoxazol-5-yl]-[1,2,4]oxadiazole (Compound No. 45),

Mass (m/z): 438.29 (M⁺+1),
-3-Methyl-5-[3-(3-cyclopentyloxy-4-methoxyphenyl)-5-methyl-4,5-dihydro-isoxazol-5-yl]-[1,2,4]oxadiazole (Compound No. 46).

Mass (m/z): 358.0 (M⁺+1),

### Example 18

### General Method of Preparation of Compound of Formula VI (wherein Y₁═Y₂═R₄═H, R₁═CH₃, Rr=CN, (CH₂)₂CN)

Oxime (Formula IV, scheme 1, 1 equiv.) and compound of Formula V (2 equiv.) were taken in tetrahydrofuran. At ambient temperature, sodium hypochloride solution was added dropwise. The reaction mixture was stirred at room temperature overnight. Tetrahydrofuran was removed under reduced pressure. Water was added, extracted with ethyl acetate, washed with saturated sodium chloride solution, dried and concentrated in vacuo. Purification was done by column chromatography using silica gel (100-200).

The following compounds were prepared following the above procedure

-3-(3-Cyclopropylmethoxy-4-difluoromethoxyphenyl)-5-methyl-4,5-dihydro-isoxazole-5-carbonitrile (Compound No. 94),

Mass (m/z): 323.25 (M⁺+1)
-3-(3-Cyclopentyloxy-4-difluoromethoxyphenyl)-5-methyl-4,5-dihydro-isoxazole-5-carbonitrile (Compound No. 95),

Mass (m/z): 337.14 (M⁺+1)
-3-(3,4-Bis-difluoromethoxyphenyl)-5-methyl-4,5-dihydro-isoxazole-5-carbonitrile (Compound No. 96),

Mass (m/z): 319.15 (M⁺+1)
-3-(3-Cyclopropylmethoxy-4-methoxyphenyl)-5-methyl-4,5-dihydro-isoxazole-5-carbonitrile (Compound No. 97),

Mass (m/z): 287.24 (M⁺+1)
-3-(3-Butoxy-4-difluoromethoxyphenyl)-5-methyl-4,5-dihydro-isoxazole-5-carbonitrile (Compound No. 98),

Mass (m/z): 325.13 (M⁺+1)
-3-(4-Difluoromethoxy-3-propoxyphenyl)-5-methyl-4,5-dihydro-isoxazole-5-carbonitrile (Compound No. 99),

Mass (m/z): 311.07 (M⁺+1)
-3-(4-Difluoromethoxy-3-isopropoxyphenyl)-5-methyl-4,5-dihydro-isoxazole-5-carbonitrile (Compound No. 100),

Mass (m/z): 311.15 (M⁺+1)
-3-[3-(Bicyclo[2.2.1]hept-2-yloxy)-4-difluoromethoxyphenyl]-5-methyl-4,5-dihydro-isoxazole-5-carbonitrile (Compound No. 101),

Mass (m/z): 363.14 (M⁺+1)
-3-(3-Benzyloxy-4-difluoromethoxyphenyl)-5-methyl-4,5-dihydro-isoxazole-5-carbonitrile (Compound No. 102),

Mass (m/z): 360.18 (M⁺+1)

### Example 19

### Preparation of 3-[3-(3-Cyclopentyloxy-4-methoxyphenyl)-5-methyl-4,5-dihydro-isoxazol-5-yl]-5-methyl-[1,2,4]oxadiazole (Compound No. 56)

To a mixture of amidoxime (100 mg, 0.00030 mole, Scheme I, Formula VII) and powdered molecular sieves (4 A°, 500 mg), dry tetrahydrofuran (3 ml) was added. The reaction mixture was stirred for about 30 minutes. Sodium hydride (11 mg, 0.0003 mole) was added and heated at about 60° C. for about 45 minutes. Methyl acetate (0.047 ml, 0.0006 mole) was added to reaction mixture and refluxed at about 65-70° C. for one hour. The reaction mixture was filtered, and washed with ethyl acetate. The organic layer was concentrated under reduced pressure to give crude product, which was purified by column chromatography using silica gel (100-200). Yield: 50 mg.

The following compounds were prepared following the above procedure

-3-[3-(3-Cyclopentyloxy-4-methoxyphenyl)-5-methyl-4,5-dihydro-isoxazol-5-yl]-5-fluoromethyl-[1,2,4]oxadiazole (Compound No. 67),

Mass (m/z): 376.22 (M⁺+1)
-3-[3-(3-Cyclopentyloxy-4-methoxyphenyl)-5-methyl-4,5-dihydro-isoxazol-5-yl]-5-(3-fluorophenyl)-[1,2,4]oxadiazol (Compound No. 70),

Mass (m/z): 438.24 (M⁺+1)
-{3-[3-(3-Cyclopentyloxy-4-methoxyphenyl)-5-methyl-4,5-dihydro-isoxazol-5-yl]-[1,2,4]oxadiazol-5-yl}-acetonitrile (Compound No. 71),

Mass (m/z): 383.24 (M⁺+1)

### Example 20

### Preparation of 3-[3-(3-Cyclopentyloxy-4-methoxyphenyl)-5-methyl-4,5-dihydro-isoxazol-5-yl]-5-(tetrahydro-furan-2-yl)-[1,2,4]oxadiazole (Compound No. 68)

Amidoxime (100 mg, 0.0003 mole, Scheme I, Formula VI) and tetrahydro-2-furoic acid (0.03 ml, 0.0003 mole) was taken in dimethylformamide (1 ml). At about 0° C., 1-hydroxybenzotriazole (40 mg, 0.0003 mole) and N-methylmorpholine (0.168 ml, 0.0012 mole) were added and stirred for about 1 hour. Thereafter, at about 0° C. 1-(3-dimethylaminopropyl)-3-ethyl carbodiimide hydrochloride (115 mg, 0.0006 mole) was added and stirred at room temperature for about 24 hours. Water (5 ml) was added, extracted with ethyl acetate. The ethyl acetate layer was dried over anhydrous sodium sulphate, and concentrated in vacuo. Dimethylformamide (2 ml) was added to the reaction residue. 50 mg powdered molecular sieves was added and refluxed at about 110-120° C. for about 4 hours. The resultant was filtered and washed with ethyl acetate. The organic layer was concentrated and purified by column chromatography using silica gel (100-200).

Yield: 40 mg

The following compounds were prepared similarly

-3-[3-(3-Cyclopentyloxy-4-methoxyphenyl)-5-methyl-4,5-dihydro-isoxazol-5-yl]-5-cyclopropyl-[1,2,4]oxadiazole (Compound No. 60),

Mass (m/z): 384.31 (M⁺+1); m.p.: 105-106° C.
-5-(3-Chlorophenyl)-3-[3-(3-cyclopentyloxy-4-methoxyphenyl)-5-methyl-4,5-dihydro-isoxazol-5-yl]-[1,2,4]oxadiazole (Compound No. 61),

Mass (m/z): 454.31 (M⁺+1); m.p.: 103-104° C.
-5-[3-(3-Cyclopentyloxy-4-methoxyphenyl)-5-methyl-4,5-dihydro-isoxazol-5-yl]-5-m-tolyl-[1,2,4]oxadiazole (Compound No. 62),

Mass (m/z): 434.42 (M⁺+1); m.p.: 131-132° C.
-3-[3-(3-Cyclopentyloxy-4-methoxyphenyl)-5-methyl-4,5-dihydro-isoxazol-5-yl]-5-(3,5-dimethyl-phenyl)-[1,2,4]oxadiazole (Compound No. 63),

Mass (m/z): 448.00 (M⁺+1); m.p.: 131° C.
-3-[3-(3-Cyclopentyloxy-4-methoxyphenyl)-5-methyl-4,5-dihydro-isoxazol-5-yl]-5-(4-fluoro-phenyl)-[1,2,4]oxadiazole (Compound No. 69),

Mass (m/z): 438.26 (M⁺+1); m.p.: 146.1-146.3° C.
-4-{3-[3-(3-Cyclopentyloxy-4-methoxyphenyl)-5-methyl-4,5-dihydro-isoxazol-5-yl]-[1,2,4]oxadiazole-5-yl)-benzonitrile (Compound No. 72),

Mass (m/z): 445.32 (M⁺+1)
-3-[3-(3-Cyclopentyloxy-4-methoxyphenyl)-5-methyl-4,5-dihydro-isoxazol-5-yl]-5-trifluoromethyl-[1,2,4]oxadiazole (Compound No. 73),

Mass (m/z): 412.25 (M⁺+1)
-3-[3-(3-Cyclopentyloxy-4-methoxyphenyl)-5-methyl-4,5-dihydro-isoxazol-5-yl]-5-(3-methoxy-phenyl)-[1,2,4]oxadiazole (Compound No. 74),

Mass (m/z): 450.26 (M⁺+1); m.p.: 121-122° C.
-3-[3-(3-Cyclopentyloxy-4-methoxyphenyl-5-methyl-4,5-dihydro-isoxazol-5-yl]-5-(3,4-dimethoxy-phenyl)[1,2,4]oxadiazole (Compound No. 75),

Mass (m/z): 480.32 (M⁺+1); m.p.: 119-120° C.
-5-Cyclopentyl-3-[3-(3-cyclopentyloxy-4-methoxyphenyl)-5-methyl-4,5-dihydro-isoxazol-5-yl]-[1,2,4]oxadiazole (Compound No. 78),

Mass (m/z): 412.33 (M⁺+1)
-3-[3-(3-Cyclopentyloxy-4-methoxyphenyl)-5-methyl-4,5-dihydro-isoxazol-5-yl]-5-(3,4-dichloro-phenyl)-[1,2,4]oxadiazole (Compound No. 79),

Mass (m/z): 488.17 (M⁺+1); m.p.: 113.5-114.9° C.

### Example 21

### Preparation of 2-[3-(3-Cyclopentyloxy-4-methoxyphenyl)-5-methyl-4,5-dihydro-isoxazol-5-yl]-pyridine (Compound No. 103)

3-cyclopentyloxy-4-methoxybenzaldehyde oxime (250 mg, 1.063 mmol) and 2-vinyl-pyridine (167 mg, 1.595 mmol) were taken in tetrahydrofuran (3 ml). The reaction mixture was stirred for about 10 minutes. Sodium hypochloride solution (1 ml, 10.63 mmol) was added gradually over 15 minutes and stirred for 2 hours. THF was evaporated off and the residue was extracted with ethylacetate. The organic layer washed with water, dried over anhydrous sodium sulphate and concentrated. The product was purified using column chromatography. Mass (m/z): 339.21 (M⁺+1).

### Example 22

### Preparation of {5-[3-(3-Cyclopentyloxy-4-methoxyphenyl)-5-methyl-4,5-dihydro-isoxazol-5-yl]-[1,3,4]thiadiazol-2-yl}-cyclopropylamine (Compound No. 49)

3-(3-cyclopentyloxy-4-methoxyphenyl)-5-methyl-4,5-dihydro-isoxazole-5-carboxylic acid (250 mg, 0.00078 mole) and cyclopropylthiosemicarbazide (102 mg, 0.00078 mole) were taken in dioxane (10 ml). At about 65° C., POCl₃ (0.07 ml, 0.00078) was added to the reaction mixture. The reaction mixture was refluxed at about 65° C. for about 5 hours and then at room temperature overnight. Dioxane was removed under reduced pressure. Saturated sodium bicarbonate solution was added. Extraction was done by ethyl acetate, and the extract washed with saturated sodium chloride solution. The product was dried over anhydrous sodium sulphate and concentrated in vacuo to give solid compound, which was further crystallized by ethanol to give white solid compound having m.pt—188-189° C. Yield=44 mg; Mass (M⁺+1)=415.

The following compound was prepared similarly

-5-[3-(3-Cyclopentyloxy-4-methoxyphenyl)-5-methyl-4,5-dihydro-isoxazol-5-yl]-[1,3,4]thiadiazol-2-yl-amine (Compound No. 48)

Mass (m/z): 375.37 (M⁺+1)

### Example 23

### Preparation of 3-(3-Cyclopentyloxy-4-methoxyphenyl)-5-(4,5-dihydro-thiazol-2-yl)-5-methyl-4,5-dihydro-isoxazole (Compound No. 51)

3-(3-Cyclopentyloxy-4-methoxyphenyl)-5-methyl-4,5-dihydroisoxazole-5-carbonitrile (70 mg, 0.0002 mole) and 2-aminoethanthiol hydrochloride (53 mg, 0.0004 mole) were taken in 5 ml ethanol. Triethylamine (0.04 ml, 0.0003 ml) was added to the reaction mixture and refluxed for about 5 hours. Ethanol was removed under reduced pressure to get crude compound, which was purified by column chromatography using silica gel (100-200). Yield: 50 mg; m.pt.: sticky solid.

### Example 24

### Preparation of 1-{5-[3-(3-Cyclopentyloxy-4-methoxyphenyl)-5-methyl-4,5-dihydro-isoxazol-5-yl]-2-ethyl-2-methyl-[1,3,4]oxadiazol-3-yl}-ethanone (Compound No. 50)

Step a: Hydrazide (100 mg, 0.0003 mole, Scheme IA, Formula VI) was taken in ethanol (5 ml). Ethylmethyl ketone (0.03 ml, 0.0004 mole) was added. The reaction mixture was stirred at refluxing temperature for about 10 hours. Ethane was removed under reduced pressure to give oily compound.

Step b: The compound from step a was taken in pyridine (3 ml). One ml acetic anhydride was added and stirred at about 100° C. for about 8 hours. A mixture of acetic anhydride and pyridine was removed under reduced pressure. 5 ml cold water was added and extraction was done by ethyl acetate. The resultant washed with saturated sodium chloride solution, dried over anhydrous sodium sulphate and concentrated in vacuo to give crude compound, which was purified by column chromatography using silica gel (100-200).

### Example 25

### Preparation of 3′-(3-Cyclopentyloxy-4-methoxyphenyl)-5,5′-dimethyl-4,5,4′,5′-tetrahydro-[3,5′]biisoxazolyl-5-carbonitrile (Compound No. 52)

Step a: Oxime (350 mg, 0.00148 mole, Scheme I, Formula IV) and methacrolein (0.73 ml, 0.0089 mole) was taken in tetrahydrofuran (10 ml). Sodium hypochlorite solution (10 ml) was added dropwise to residue mixture. The reaction mixture was stirred at room temperature for about 14-16 hours. Tetrahydrofuran was removed under reduced pressure. Water (10 ml) was added, extracted with ethyl acetate, washed with saturated sodium chloride solution, dried over anhydrous sodium sulphate and concentrated in vacuo to give oily compound.

Step b: The compound from step a was taken in ethanol (10 ml) and to it hydroxylamine hydrochloride (160 mg, 0.0023 mole) and anhydrous sodium acetate (189 mg, 0.0028 mole) were added. The reaction mixture was stirred at room temperature for about one and half an hours. Ethanol was removed under reduced pressure, water was added, extracted with ethyl acetate. Dried over anhydrous sodium sulphate and concentrated in vacuo to give oily compound.

Step c:—The compound from step b was taken in tetrahydrofuran (5 ml) and to it methacrylonitrile (0.246 ml, 0.0036 mole) was added. Sodium hypochloride solution (3 ml) was added to reaction mixture dropwise within a 15 minute interval. The reaction mixture was stirred for about 15-16 hours. Tetrahydrofuran was removed under reduced pressure. Water (30 ml) was added, extracted with ethyl acetate. Dried over anhydrous sodium sulphate and concentrated in vacuo to give oily compound, which was purified by column chromatography using silica gel (100-200). Yield: 50 mg; m.pt: oily.

### Example 26

### Preparation of 5-[3-(3-Cyclopentyloxy-4-methoxyphenyl)-5-methyl-4,5-dihydro-isoxazol-5-yl]-3H-[1,3,4]oxadiazole-2-thione (Compound No. 55)

Hydrazide (70 mg, 0.0002 mole, Scheme IA, Formula VIII) was taken in ethanol (5 ml). To it potassium hydroxide solution (0.11 g, 0.0002 mole) in 1 ml ethanol were added followed by carbon disulfide (1 ml). The reaction mixture was refluxed for about 8 hours. Ethanol was removed under reduced pressure. The reaction mixture was neutralized by dilute hydrochloride (2N), and extracted with ethyl acetate. The resultant washed with saturated sodium chloride solution, dried over anhydrous sodium sulphate and concentrated in vacuo to give crude product, which was purified by column chromatography using silica gel 100-200. Yield: 70 mg; m.pt: 195.5-200° C.

### Example 27

### Preparation of 4-Amino-5-[3-(3-cyclopentyloxy-4-methoxyphenyl)-5-methyl-4,5-dihydro-isoxazol-5-yl]-4H-[1,2,4]triazole-3-thiol (Compound No. 54)

A mixture of 5-[3-(3-Cyclopentyloxy-4-methoxyphenyl)-5-methyl-4,5-dihydro-isoxazol-5-yl]-3H-[1,3,4]oxadiazole-2-thione (50 mg, 0.00013 mole, Example 26) and hydrazine hydrate (0.02 ml, 0.0003 mole) in ethanol (2 ml) were refluxed for about 6 hours. The solvent and excess hydrazine hydrate were removed under reduced pressure. Water was added, and the aqueous phase was extracted with ethyl acetate. The extract washed with brine, dried over anhydrous sodium sulphate and concentrated in vacuo to give crude product, which was recrystallized using ethyl acetate hexane (20:80). Yield: 15 mg; m.pt.: 228-229° C.

### Example 28

### Preparation of Acetic acid (Z)-2-amino-2-{3-[3-(cyclopentyloxy)-4-methoxyphenyl]-5-methyl-4,5-dihydroisoxazol-5-yl }vinyl ester (Compound No. 105)

[3-(3-Cyclopentyloxy-4-methoxyphenyl)-N-hydroxy-5-methyl-4,5-dihydroisoxazole-5-carboxamidine (Example 4, 0.0007 mole, 250 mg) was dissolved in dichloromethane. To it acetic anhydride (0.0007 mole, 0.07 ml) and triethyl amine (0.0007 mole, 0.105 ml) were added. The reaction mixture was stirred at an ambient temperature for about 2 hours. The mixture washed with water. The organic layer was dried over anhydrous sodium sulphate, concentrated in vacuo and the residue was purified over column chromatography. Yield: 46%; m.pt.: 130.9° C.; ¹H NMR (CDCl₃): δ 7.29-7.30 (d, 1H), 7.036-7.06 (d, 1H), 6.83-6.86 (d, 1H), 5.259 (s, 2H), 4.78-4.81 (m, 1H), 3.98 (d, 1H), 3.87 (s, 3H), 3.276-3.33 (d, 1H), 2.16 (s, 3H), 1.79-2.09 (m, 8H), 1.25-1.29 (m, 3H); Mass (m/z): 376.24 (M⁺+1).

### Example 29

### Preparation of 3-{3-[3-(cyclopentyloxy)-4-methoxyphenyl]-5-methyl-4,5-dihydroisoxazol-5-yl}-5-methyl-1,4,2-dioxazole-5-carboxylic acid ethyl ester (Compound No. 53)

To a solution of 3-(3-cyclopentyloxy-4-methoxyphenyl)-5-methyl-4,5-dihydro-isoxazole-5-carbaldehyde oxine (0.480 g, 1.5116 mmole) and 2-oxo propionic acid ethyl ester (1.052 g, 9.0698 mmole) in tetrahydrofuran was added bleach over a period of about 40 minutes. The reaction mixture was allowed to stir at room temperature for about one and half hours. Thereafter, tetrahydrofuran was removed over buchi. To the residue was added water (20 mL) and the resulting solution was extracted with ethyl acetate. Ethyl acetate layer was dried over anhydrous sodium sulphate and finally concentrated to afford an oily residue, which was purified by column chromatography. Yield: 0.200 g; m.p: 139-140° C.; Mass (m/z): 376.24 (M⁺+1).

### Example 30

### Efficacy of Compounds as PDE IV Inhibitors PDE-IV Enzyme Assay

The efficacy of compounds of PDE-4 inhibitors was determined by an enzyme assay using U937 cell cytosolic fraction (BBRC, 197: 1126-1131, 1993). Hydrolysis of cAMP to AMP was monitored using HPLC and [³H]cAMP in the sample was detected using FLO-ONE Detector.

The enzyme preparation was incubated in the presence and absence of the test compound for 30 min and amount, [³H]cAMP measured in the sample. The IC₅₀ values were found to be in the range of double-digit nM to >10 μM concentration.

1. Compounds having the structure of Formula I: 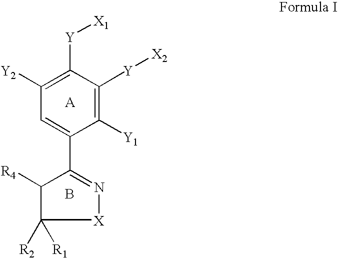
their pharmaceutically acceptable salts, pharmaceutically acceptable solvates, enantiomers, diastereomers or N-oxides wherein
1) when X is oxygen in Formula I;
R₁ is selected from: hydrogen; alkyl; alkenyl; alkynyl; cycloalkyl; cyano; nitro; amino; substituted amino; hydroxyl; alkoxy; aryloxy, COR′; COOR′
(wherein R′ can be hydrogen, alkyl, alkenyl, alkynyl, (un)saturated cycloakyl, aryl, aralkyl, heterocyclyl, (heterocyclyl)alkyl, or (heteroaryl)alkyl);
aryl; aralkyl; heteroaryl; heterocyclyl; (heteroaryl) alkyl; (heterocycyl) alkyl; (CH₂)₁₋₄OR′ (wherein R′ is a defined above, but also including hydroxy); C(═O)NRₓR_{y}
(wherein Rₓ and R_{y} can be independently selected from hydrogen, alkyl, C₃₋₆ alkenyl, C₃₋₆ alkynyl, (un)saturated cycloalkyl aryl, aralkyl, heteroaryl, heterocyclyl, heteroarylalkyl, or heterocyclylalkyl); or
(CH₂)C(═O)R₃
[wherein m is an integer in the range of 0-2 and R₃ can be optionally substituted Rₚ or R_{q} (wherein Rₚ can be a 4-12 membered (un)saturated monocyclic or bicyclic ring containing, 1-4 heteroatom(s) selected from N, O and S wherein the ring can be attached to (CH₂)ₘC(═O) through N and R_{q} can be a 4-12 membered (un)saturated monocyclic or bicyclic ring containing 0-4 heteroatom(s) selected from the group consisting of N, O and S wherein the ring can be attached to (CH₂)ₘC(═O) through C) and wherein the substituents of R₃ can be one or more of:
alkyl, alkenyl, alkynyl, (un)saturated cycloalkyl, halogen, hydroxyl alkoxy, aryloxy, nitro, cyano, amino, substituted amino hydroxyalkyl, oxo, acyl, optionally substituted amino (wherein the substituents are selected from C₁-C₆ alkyl, aryl, aralkyl, or cycloalkyl), aryl, carboxyl, alkaryl, carbamoyl, alkyl ether, C(═O)NR₅R₆ (wherein R₁ and R₆ are independently selected from hydrogen, alkyl, C₃₋₆ alkynyl, aryl, and aralkyl), optionally substituted monocyclic or bicyclic 4-12 membered carbocyclic ring system (wherein the optional substituents(s) is/are selected from alkyl, alkenyl, alkynyl, halogen, hydroxy, and alkoxy), heteroaryl, heterocyclyl, heteroarylalkyl, or
heterocyclylalkyl];
R₂ is selected from: cyano; heteroaryl; heterocycyl; or (CH₂)ₙNHCOR₇ (wherein n represents an integer 1 to 6 and R₇ can represent hydrogen, alkyl, alkenyl, alkynyl, (un)saturated, cycloalkyl, alkoxy, aryloxy, aryl, aralkyl, heteroaryl, heterocyclyl, (CH₂)₁₋₄OR′ wherein R′ is the same as defined above, or NRₓR_{y} wherein Rₓ and R_{y} are the same as defined above);
R₄ is selected from: hydrogen; alkyl; halogen; cyano; carboxy; or R(═O)NRₓR_{y} wherein it Rₓ and R_{y} are the same as defined above;
X₁ and X₂ are independently selected from: hydrogen; alkyl; alkenyl; alkynyl; cycloalkyl; acyl; aryl; aralkyl; heteroaryl; heterocyclyl; (heteroaryl)alkyl; or (heterocyclyl)alkyl;
Y is selected from: an oxygen atom; a sulphur atom; or NR
(wherein R is selected from hydrogen, alkyl, alkenyl alynyl, un(saturated) cycloalkyl, acyl, aryl, aralkyl, heteroaryl, heterocyclyl, (heteroaryl)alkyl, or
(heterocyclyl)alkyl);
Y₁ and Y₂ are independently selected from: hydrogen; alkyl; nitro; cyano; halogen; OR wherein R is the same as defined earlier; SR wherein R is the same as defined earlier; NHR wherein is the same as defined earlier; COOR′; or COR′ wherein R′ is the same as defined above, or further Y₁ and X₂, X₁ and Y₂, X₁ and X₂ may together form a ring fused with the ring A containing 3-5 carbon atoms within the ring and having 1-3 heteroatoms selected from N, O or S; and
2) when X is NR₈ or S wherein R₈ is hydrogen, lower alkyl (C₁-C₆) or aryl;
R₁, R₄, X₁, X₂, Y, Y₁ and Y₂ are the same as defined above;
R₂ is selected from: (CH)ₙNHCOR₇ (wherein n represents an integer 1 to 6 and R₇ is the same as defined above),
with the provisio that when R₂ is heterocyclyl, R₁ can not be (CH₂)₁₋₄OR′, C(═O)NRₓR_{y} or (CH₂)ₘ—C(═O)R₃.

2. A compound having the structure of Formula XXXIV, 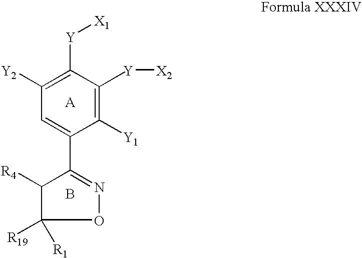
their pharmaceutically acceptable salts, pharmaceutically acceptable solvates enantiomers, diastereomers or N-oxides
wherein
R₁ is selected from: hydrogen; alkyl; alkenyl; alkynyl; cycloalkyl; cyano; nitro; amino; substituted amino; hydroxyl; alkoxy; aryloxy; COR′; COOR′
(wherein R′ can be hydrogen, alkyl, alkenyl, alkynyl, (un)saturated cycloalkyl, aryl; aralkyl, heterocyclyl, heterocyclyl; (heteroaryl) alkyl, or (heterocyclyl)alkyl);
aryl; aralkyl; heteroaryl; heterocyclyl; (heteroaryl) alkyl; (heterocyclyl) alkyl); (CH₂)₁₋₄OR′ (wherein R′ is as defined above, but also including hydroxy); C(O)NRₓR_{y}
(wherein Rₓ and R_{y} can be independently selected from hydrogen, alkyl, C₃₋₆ alkenyl, C₃₋₆ alkynyl, (un)saturated cycloalkyl, aryl, aralkyl, heteroaryl, heterocyclyl, heteroarylalkyl, or heterocyclylalkyl); or
(CH₂)ₘ—C(═O)R₃
[wherein m is an integer in the range of 0-2 and R₃ can be optionally substituted Rₚ or R_{q} (wherein R₁ can be a 4-12 membered (un)saturated monocyclic or bicyclic ring containing 1-4 heteroatoms(S) selected from N, O and S wherein the ring can be attached to (CH₂)ₘC(═O) through N and R_{q} can be a 4-12 membered (un)saturated monocyclic or bicyclic ring containing 0-4 heteroatom(s) selected from the group consisting of N, O and S wherein the ring can be attached to (CH₂)ₘC(═O) through C) and wherein the substituents of R₃ can be one or more of:
alkyl, alkenyl, alkynyl, (un)saturated cycloalkyl, halogen, hydroxyl, alkoxy, aryloxy), nitro, cyano, amino, substituted amino, hydroxyalkyl, oxo, acyl, optionally substituted amino (wherein the substituted are selected from C₁-C₆ alkyl, aryl, aralkyl, or cycloalkyl) aryl, carboxyl, alkaryl, carbamoyl, alkyl ether, C(═O)—NR₅R₆ (wherein R₅ and R₆ are independently selected from hydrogen, alkyl, C₃₋₆ alkenyl, C₃₋₆ alkynyl, aryl, and aralkyl), optionally substituted monocyclic or bicyclic 4-12 membered carbocyclic ring system (wherein the optional substituent(s) is/are selected from, alkyl, alkenyl, alkynyl, halogen, hydroxyl and alkoxy), heteroaryl, heterocyclyl, heteroarylalkyl, or
heterocyclylalkyl];
R₄ is selected from: hydrogen; alkyl; halogen; cyano; carboxy; or C(═O)NRₓR_{y}, wherein Rₓ and R_{y} are the same as defined above;
X₁ and X₂ are independently selected from: hydrogen; alkyl; alkenyl, alkynyl; cycloalkyl; acyl; aryl; aralkyl; heteroaryl; heterocyclyl; (heteroaryl)alkyl; or (heterocyclyl) alkyl;
Y is selected from an oxygen atom; a sulphur atom; or NR
(wherein R is selected from hydrogen, alkyl, alkenyl, alkynyl, (un)(saturated) cycloalkyl, acyl, aryl, aralkyl, heteroaryl, heterocyclyl, (heteroaryl)alkyl, or
(heterocyclyl)alkyl);
Y₁ and Y₂ are independently selected from hydrogen; alkyl; nitro; cyano; halogen; OR wherein R is the same as defined earlier; SR wherein R is the same as defined earlier; NHR wherein R is the same as defined earlier; COOR′; or COR′ wherein R′ is the same as defined above or further, Y₁ and X₂, X₁ and Y₂, X₁ and X₂ may together form a ring fused with the ring, A containing 3-5 carbon atoms within the ring and having 1-3 heteroatoms selected from N, O or S; and
R₁₉ represents —CONHNH₂, or 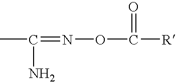
wherein R′ is the same as defined for Formula I.

3. The compound of claim 1 having the structure of Formula XXXII, 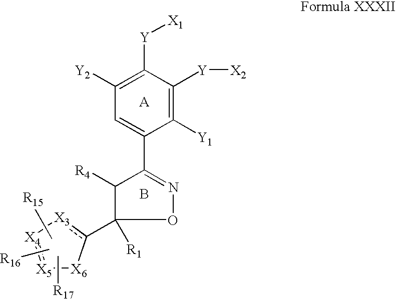
their pharmaceutically acceptable salts, pharmaceutically acceptable solvates, enantiomers, diastereomers or N-oxides wherein
wherein
R₁ is selected from: hydrogen; alkyl, alkenyl; alkynyl; cycloalkyl, cyano; nitro; amino; substituted amino; hydroxyl; alkoxy; aryloxy; COR′; COOR′
(wherein R′ can be hydrogen, alkyl, alkenyl, alkynyl, (un)saturated cycloalkyl, aryl aralkyl; heterocyclyl, (heterocyclyl)alkyl, or (heteroaryl)alkyl);
aryl; aralkyl; heteroaryl; heterocyclyl; (heteroaryl) alkyl; (heterocyclyl) alkyl; (CH₂)₁₋₄OR′ (wherein R′ is as defined above, but also including hydroxy); C(═O)NRₓR_{y}
(wherein Rₓ and R_{y} can be independently selected from hydrogen, alkyl, C₃₋₆ alkenyl, C₃₋₆ alkynyl, (un)saturated cycloalkyl, aryl, aralkyl, heteroaryl, heterocyclyl, heteroarylalkyl, or heterocyclylalkyl); or
(CH₂)ₘ—C(═O)R₃
[wherein m is an integer in the range of 0-2 and R₃ can be optionally substituted Rₚ or R_{q} (wherein Rₚ can be a 4-12 membered (un)saturated monocyclic or bicyclic ring containing 1-4 heteroatom(s) selected from N, O and S wherein the ring can be attached to (CH₂)ₘC(═O) through N and R_{q} can be a 4-12 membered (un)saturated monocyclic or bicyclic ring containing 0-4 heteroatom(s) selected from the group consisting of N, O and S wherein the ring can be attached to (CH₂)ₘC(═O) through C) and, wherein the substituents of R₃ can be one or more of:
alkyl, alkenyl, alkynyl, (un)saturated cycloalkyl, halogen, hydroxyl, alkoxy, aryloxy, nitro, cyano, amino, substituted amino, hydroxyalkyl, oxo, acyl, optionally substituted amino (wherein the substituents are selected from C₁-C₆ alkyl, aryl, aralkyl, or cycloalkyl), aryl, carboxyl, alkaryl, carbamoyl, alkyl ether, C(═O)NR₅R₆ (wherein R₅ and R₆ are independently selected from hydrogen, alkyl C₃₋₆ alkenyl, alkynyl, aryl, and aralkyl) optionally substituted monocyclic or bicyclic, 4-12 membered carbocyclic ring system (wherein the optional substituted(s) is/are selected from alkyl, alkenyl, alkynyl, halogen, hydroxyl, and alkoxy), heteroaryl, heterocyclyl, heteroarylalkyl, or
heterocyclylalkyl];
R₄ is selected from: hydrogen; alkyl; halogen; cyano; carboxy; or C(═O)NRₓR_{y} wherein Rₓ and R_{y} are the same as defined above;
Y is selected from: an oxygen atom; a sulphur atom; or NR
(wherein R is selected from hydrogen, alkyl, alkenyl, alkynyl, un(saturated) cycloalkyl, acyl, aryl, aralkyl, heteroaryl, heterocycyl, (heteroaryl)alkyl, or
(heterocyclyl)alkyl);
Y₁ and Y₂ are independently selected from: hydrogen alkyl; nitro; cyano; halogen; OR wherein R is the same as defined earlier; SR wherein R is the same as defined earlier; NHR wherein R is the same as defined earlier; COOR′; or COR′ wherein R′ is the same as defined above, or further, Y₁ and X₂, X₁ and Y₂, X₁ and X₂ may together form a ring fused with the ring A containing 3-5 carbon atoms within the ring and having 1-3 heteroatoms selected from N, O or S;
X₁ represents alkyl;
X₂ represents alkyl, cycloalkyl or aralkyl;
X₃, X₄, X₅ and X₆ independently represent C, CH, CH₂, CO, CS, NH, O, S; R₁₅, R₁₆, and R₁₇ independently represent no atom, COCH₃, COOC₂H₅, NH₂, NH-cyclopropyl, CN, SH; and
---- represents an optional single bond.

4. The compound of claim 1 having the structure of Formula XXIII, 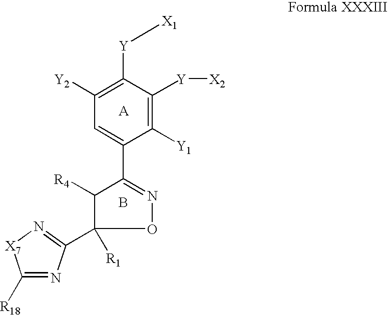
their pharmaceutically acceptable salts, pharmaceutically acceptable solvates, enantiomers, diastereomers or N-oxides wherein
wherein
R₁ is selected from: hydrogen; alkyl; alkenyl; cycloalkyl; cyano; nitro; amino;
substituted amino, hydroxyl; alkoxy; aryloxy; COR′; COOR′
(wherein R′ can be hydrogen, alkyl, alkenyl, alkynyl, (un)saturated cycloalkyl, aryl, aralkyl, heterocyclyl, (heterocyclyl)alkyl, or (heteroaryl)alkyl);
aryl; aralkyl; heteroaryl; heterocyclyl; (heteroaryl) alkyl; (heterocyclyl) alkyl; (CH₂)₁₋₄OR′ (wherein R′ is as defined above, but also including hydroxy); C(═O)NRₓR_{y}
(wherein Rₓ and R_{y} can be independently selected from hydrogen, alkyl, C₃₋₆ alkenyl, C₃₋₆ alkynyl, (un)saturated cycloalkyl, aryl, aralkyl, heteroaryl, heterocyclyl, heteroarylalkyl, or heterocyclylalkyl); or
(CH₂)ₘ—C(═O)R₃
[wherein m is an integer in the range of 0-2 and R₃ can be optionally substituted Rₚ or R_{q} (wherein Rₚ can be a 4-12 membered (un)saturated monocyclic or bicyclic ring containing 1-4 heteroatom(s) selected from N, O and S wherein the ring can be attached to (CH₂)ₘC(═O) through N and R_{q} can be a 4-12 membered (un)saturated monocyclic or bicyclic ring containing 0-4 heteroatom(s) selected from the group consisting of N, O and S wherein the ring can be attached to (CH₂)ₘC(═O) through C) and wherein the substituents of R₃ can be one or more of:
alkyl, alkenyl, alkynyl, (un)saturated cycloalkyl, halogen, hydroxyl, alkoxy, aryloxy, nitro, cyano, amino, substituted amino, hydroxyalkyl, oxo, acyl, optionally substituted amino (wherein the substituents are selected from C₁-C₆ alkyl, aryl, aralkyl, or cycloalkyl), aryl, carboxyl, alkaryl, carbamoyl, alkyl ether, C(═O)NR₅R₆ (wherein R₅ and R₆ are independently selected from hydrogen, alkyl, C₃₋₆ alkenyl, C₃₋₆ alkynyl, aryl, and aralkyl), optionally substituted monocyclic or bicyclic 4-12 membered carbocyclic ring system (wherein the optional substituent(s) is/are selected from alkyl, alkenyl, alkynyl, halogen, hydroxyl, and alkoxy), heteroaryl heterocyclyl, heteroarylalkyl, or
heterocyclylalkyl];
R₄ is selected from: hydrogen; alkyl; halogen; cyano; carboxy; or C(═O)NRₓR_{y} wherein Rₓ and R_{y} are the same as defined above;
X₁ and X₂ are independently selected from: hydrogen; alkyl; alkenyl; alkynyl; cycloalkyl; acyl; aryl; aralkyl; heteroaryl; heterocyclyl; (heteroaryl)alkyl; or (heterocyclyl)alkyl;
Y is selected from: an oxygen atom; a sulphur atom; or NR
(wherein R is selected is hydrogen, alkyl alkenyl, alkynyl, un(saturated) cycloalkyl, acyl, aryl, aralkyl, heteroaryl, heterocyclyl, (heteroaryl) alkyl, or
(heterocyclyl)alkyl);
Y₁ and Y₂ are independently selected from: hydrogen; alkyl; nitro; cyano; halogen; OR wherein R is the same as defined earlier; SR wherein R is the same as defined earlier; NHR wherein R is the same as defined earlier; COOR′; or COR′ wherein R′ is the same as defined above, or further, Y₁ and X₂, X₁ and Y₂, X₁ and X₂ may together form a ring fused with the ring A containing 3-5 carbon atoms within the ring and having 1-3 heteroatoms selected from N, O or S;
X₇ represents O or S; and
R₁₈ represent hydrogen, alkyl, aryl, heteroaryl, cycloalkyl or heterocyclyl.

5. The compound of claim 1 wherein R₂ is cyano.

6. The compound of claim 1 wherein R₂ is (CH₂)ₙNHCOR₇, n represents an integer 1 to 6; and R₇ can represent hydrogen, alkyl, alkenyl, alkynyl, (un)saturated, cycloalkyl, alkoxy, aryloxy, aryl, aralkyl, heteroaryl, heterocyclyl, (CH₂)₁₋₄OR′ wherein R′ is the same as defined above, or NRₓR_{y} (wherein Rₓ and R_{y} can be independently selected from hydrogen, alkyl, C₃₋₆alkenyl, C₃₋₆ alkynyl, (un)saturated Cycloalkyl, aryl, aralkyl, heteroaryl, heterocyclyl, heteroarylalkyl, or heterocyclylalkyl).

7. The compound of claim 1 wherein R₂ is 6-membered heteroaryl.

8. A pharmaceutical composition comprising a therapeutically effective amount of a compound of claim 1, together with at least one pharmaceutically acceptable carrier, excipient or diluent.

9. A method for treating, preventing, inhibiting or suppressing an inflammatory condition or disease in a patient, comprising administering to the said patient a therapeutically effective amount of a compound of claim 1.

10. A method for treating, preventing, inhibiting or suppressing an inflammatory condition or disease in a patient, comprising administering to the said patient a therapeutically effective amount of a pharmaceutical composition of claim 8.

11. A method for the treatment, prevention, inhibition or suppression of AIDS, asthma, arthritis, bronchitis, chronic obstructive pulmonary disease (COPD), psoriasis, allergic rhinitis, shock, atopic dermatitis, crohn's disease, adult respiratory distress syndrome (ARDS), eosinophilic granuloma, allergic conjunctivitis, osteoarthritis, ulcerative colitis and other inflammatory diseases in a patient comprising administering to said patient a therapeutically effective amount of a compound of claim 1.

12. A method for the treatment, prevention, inhibition or suppression of AIDS, asthma, arthritis, bronchitis, chronic obstructive pulmonary disease (COPD), psoriasis, allergic rhinitis, shock, atopic dermatitis, crohn's disease, adult respiratory distress syndrome (ARDS), eosinophilic granuloma, allergic conjunctivitis, osteoarthritis, ulcerative colitis and other inflammatory diseases in a patient comprising administering to said patient a therapeutically effective amount of a pharmaceutical composition of claim 8.

13. A method for the preparation of compounds of Formula VII (a), 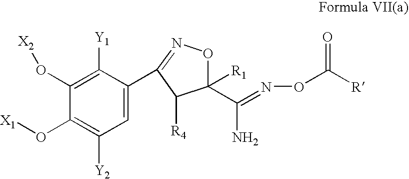
their pharmaceutically acceptable salts; pharmaceutically acceptable solvates, enantiomers, diastereomers or N-oxides, the method comprising:
reacting a compound of Formula II 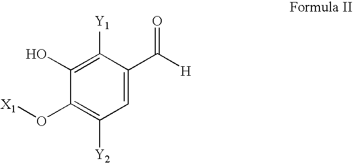
with a compound of Formula X₂Z (wherein Z is halogen) to give a compound of Formula III, wherein 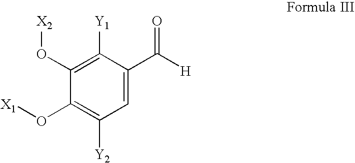
X₁ and X₂ are independently selected from: alkyl; alkenyl; alkynyl; cycloalkyl; acyl; aryl; aralkyl; heteroaryl; heterocyclyl; (heteroaryl)alkyl; or (heterocyclyl)alkyl;
Y₁ and Y₂ are independently selected from: hydrogen; alkyl; nitro; cyano; halogen; OR wherein R is the same as defined earlier; SR wherein R is the same as defined earlier; NHR wherein R is the same as defined earlier; COOR′; or COR′ wherein R′ is the same as defined above, or further, Y₁ and X₂, X₁ and Y₂, X₁ and X₂ may together form a ring fused with the ring A containing 3-5 carbon atoms within the ring and having 1-3 heteroatoms selected from N, O or S;
reacting the compound of Formula III with hydroxylamine hydrochloride to give a compound of Formula IV; 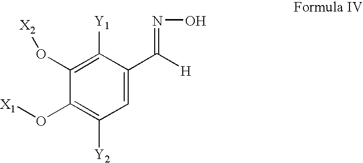
treating the compound of Formula IV with a compound of Formula V to give a compound of Formula VI 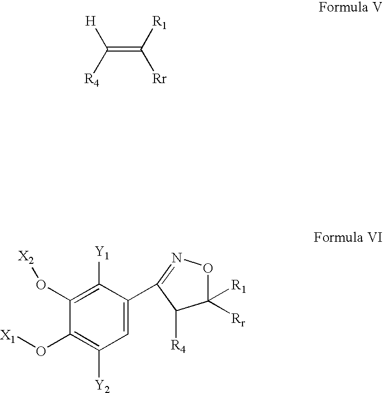
wherein
R₁ is selected from: hydrogen; alkyl; alkenyl, alkynyl; cycloalkyl; cyano; nitro; amino; substituted amino; hydroxyl; alkoxy; aryloxy; COR′; COOR′
(wherein R′ can be hydrogen, alkyl, alkenyl, alkynyl, (un)saturated cycloalkyl, aryl, aralkyl, heterocyclyl, (heterocyclyl)alkyl, or (heteroaryl)alkyl);
aryl; aralkyl; heteroaryl; heterocyclyl; (heteroaryl) alkyl; (heterocyclyl) alkyl; (CH₂)₁₋₄OR′ (wherein R′ is as defined above, but also including hydroxy); C(═O)NRₓR_{y}
(wherein Rₓ and R_{y} can be independently selected from hydrogen, alkyl, C₃₋₆ alkenyl, C₃₋₆ alkynyl (un)saturated cycloalkyl, aryl, aralkyl, heteroaryl heterocyclyl, heteroarylalkyl, or heterocyclylalkyl); or
(CH₂)ₘ—C(═O)R₃
[wherein m is an integer in the range of 0-2 and R₃ can be optionally substituted Rₚ or R_{q} (wherein Rₚ can be a 4-12 membered (un)saturated monocyclic or bicyclic ring 1-4 heteroatom(s) selected from N, O and S wherein the ring can be attached to (CH₂)ₘC(═O) through N and R_{q} can be a 4-12 membered (un)saturated monocyclic or bicyclic ring containing 0-4 heteroatom(s) selected from the group consisting of N, O and S wherein the ring can be attached to (CH₂)ₘC(═O) through C) and wherein the substituents of R₃ can be one or more of:
alkyl, alkenyl, alkynyl, (un)saturated cycloalkyl, halogen, hydroxyl, alkoxy, aryloxy, nitro, cyano, amino, substituted amino, hydroxyalkyl, oxo, acyl, optionally substituted amino (wherein the substituents are selected from C₁-C₆ alkyl, aryl, aralkyl, or cycloalkyl), aryl, carboxyl, alkaryl, carbamoyl, alkyl ether, C(═O)NR₅R₆ (wherein R₅ and R₆ are independently selected from hydrogen, alkyl, C₃₋₆ alkenyl, C₃₋₆ alkynyl, aryl, and aralkyl), optionally substituted monocyclic or bicyclic 4-12 membered carbocyclic ring system (wherein the optional substituent(s) is/are selected from alkyl, alkenyl, alkynyl, halogen, hydroxyl, and alkoxy), heteroaryl, heterocyclyl, heteroarylalkyl, or
heterocyclylalkyl];
R₄ is selected from: hydrogen; alkyl; halogen; cyano; carboxy; or C(═O)NRₓR_{y} wherein Rₓ and R_{y} are the same as defined above;
and Rr represents [(CH₂)ₙCN, COOH, COOCH₃, CHO or pyridyl, wherein is 0 to 2)];
reacting the compound of Formula VI with hydroxylamine hydrochloride (when Rr is CN) to give a compound of Formula VII; and 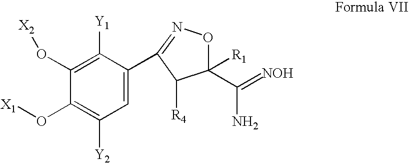
reacting the compound of Formula VII with a compound of Formula (R′CO)₂O to give the compound of Formula VII(a) (wherein R′ can be hydrogen, alkyl, alkenyl, alkynyl, (un)saturated cycloalkyl, aryl, aralkyl, heterocyclyl, (heterocyclyl)alkyl, or (heteroaryl)alkyl).

14. (canceled)

15. (canceled)

16. (canceled)

17. (canceled)

18. (canceled)

19. (canceled)

20. (canceled)

21. A method for the preparation of compounds of Formula XX, 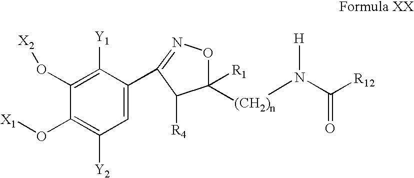
their pharmaceutically acceptable salts, pharmaceutically acceptable solvates, enantiomers, diastereomers or N-oxides,
wherein
R₁ is selected from: hydrogen; alkyl; alkenyl; alkynyl; cycloalkyl; cyano; nitro; amino; substituted amino; hydroxyl; alkoxy; aryloxy; COR′; COOR′
(wherein R′ can be hydrogen, alkyl, alkenyl, alkynyl, (un)saturated cycloalkyl, aryl, aralkyl, heterocyclyl, (heterocyclyl)alkyl, or (heteroaryl)alkyl);
aryl; aralkyl; heteroaryl; heterocyclyl; (heteroaryl) alkyl; (heterocyclyl) alkyl; (CH₂)₁₋₄OR′ (wherein R′ is as defined above, but also including hydroxy); C(═O)NRₓR_{y}
(wherein Rₓ and R_{y} can be independently selected from hydrogen, alkyl, C₃₋₆ alkenyl, C₃₋₆ alkynyl, (un)saturated cycloalkyl, aryl, aralkyl, heteroaryl, heterocyclyl, heteroarylalkyl, or heterocyclylalkyl); or
(CH₂)ₘ—C(═O)R₃
[wherein m is an integer in the range of 0-2 and R₃ can be optionally substituted Rₚ or R_{q} (wherein Rₚ can be a 4-12 membered (un)saturated monocyclic or bicyclic ring containing 1-4 heteroatom(s) selected from N, O and S wherein the ring can be attached to (CH₂)ₘC(═O) through N and R_{q} can be a 4-12 membered (un)saturated monocyclic or bicyclic ring containing 0-4 heteroatom(s) selected from the group consisting of N, O and S wherein the ring can be attached to (CH₂)ₘC(═O) through C) and wherein the substituents of R₃ can be one or more of:
alkyl, alkenyl, alkynyl (un)saturated cycloalkyl, halogen, hydroxyl, alkoxy, aryloxy, nitro, cyano, amino, substituted amino, hydroxyalkyl, oxo acyl, optionally substituted amino (wherein the substituents are selected from C₁-C₆ alkyl, aryl, aralkyl, or are independently selected from hydrogen, alkyl C₃₋₆ alkenyl, C₃₋₆ alkynyl, aryl, and aralkyl), optionally substituted monocyclic or bicyclic 4-12 membered carbocyclic ring system (wherein the optional substituents(s) is/are selected from alkyl, alkenyl, alkynyl, halogen, hydroxyl, and alkoxy), heteroaryl, heterocyclyl, heteroarylalkyl, or
heterocyclylalkyl];
R₄ is selected from: hydrogen; alkyl; halogen; cyano; carboxy; or C(═O)NRₓR_{y} wherein Rₓ and R_{y} are the same as defined above;
X₁ and X₂ are independently selected from: hydrogen; alkyl; alkenyl; alkynyl; cycloalkyl; acyl; aryl; aralkyl; heteroaryl; heterocyclyl; (heteroaryl)alkyl; or (heterocyclyl)alkyl;
Y₁ and Y₂ are independently selected from: hydrogen; alkyl; nitro; cyano; halogen; OR wherein R is the same as defined earlier; SR wherein R is the same as defined earlier; NHR wherein R is the same as defined earlier; COOR′; or COR′ wherein R′ is the same as defined above, or further, Y₁ and X₂, X₁ and Y₂, X₁ and X₂ may together form a ring fused with the ring A containing 3-5 carbon atoms within (the ring and having 1-3 heteroatom selected from N, O or S; and
R₁₂ is alkyl, cycloalkyl, aryl, heteroaryl or heterocyclyl;
the method comprising:
reacting a compound of Formula IV with a compound of Formula XVI 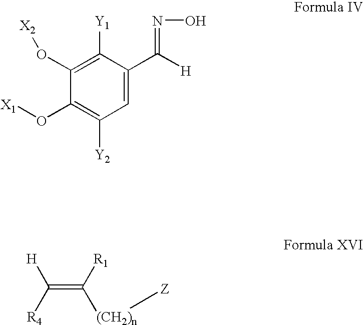
to give a compound of Formula XVII; 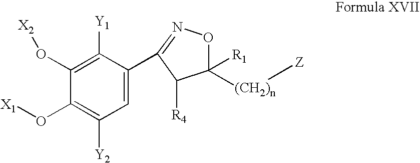
treating the compound of Formula XVII with potassium phthalamide to give a compound of Formula XVIII; 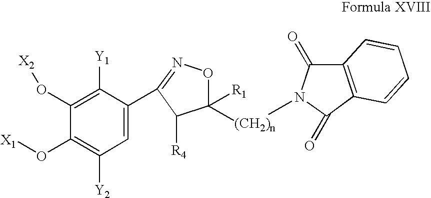
treating the compound of Formula XVIII with a hydrazine hydrate to give a compound of Formula XIX; and 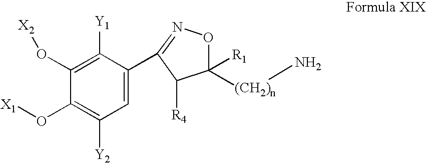
treating the compound of Formula XIX with a compound of Formula R₁₂COCl or R₁₂COOH to give the compound of Formula XX.

22. A method for the preparation of compounds of Formula XXIII, 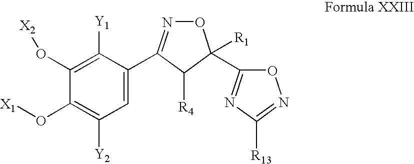
their pharmaceutically acceptable salts, pharmaceutically acceptable solvates, enantiomers, diastereomers or N-oxides,
wherein
R₁ is selected from: hydrogen; alkyl; alkenyl; alkynyl; cycloalkyl; cyano; nitro; amino; substituted amino; hydroxyl; alkoxy; aryloxy; COR′; COOR′
(wherein R′ can be hydrogen, alkyl, alkenyl, alkynyl, (un)saturated cycloalkyl, aryl, aralkyl, heterocyclyl, (heterocycyl)alkyl, or (heteroaryl)alkyl);
aryl; aralkyl; heteroaryl; heterocycyl; (heteroaryl) alkyl; (heterocyclyl) alkyl; (CH₂)₁₋₄OR′ (wherein R′ is as defined above, but also including hydroxy); C(═O)NRₓR_{y}
(wherein Rₓ and R_{y} can be independently selected from hydrogen, alkyl, C₃₋₆ alkenyl, C₃₋₆ alkynyl, (un)saturated cycloalkyl, aryl, aralkyl, heteroaryl, heterocyclyl, heteroarylalkyl, or heterocyclylalkyl); or
(CH₂)ₘ—C(═O)R₃
[wherein m is an integer in the range of 0-2 and R₃ can be optionally substituted Rₚ or R_{q} (wherein Rₚ can be a 4-12 membered (un)saturated monocyclic or bicyclic ring containing 1-4 heteroatom(s) selected from N, O and S wherein the ring can be attached to (CH₂)ₘC(═O) through N and R_{q} can be a 4-12 membered (un)saturated monocyclic or bicyclic ring containing 0-4 heteroatom(s) selected from the group consisting of N, O and S wherein the ring can be attached to (CH₂)ₘC(═O) through C) and wherein the substituents of R₃ can be one or more of:
alkyl, alkenyl, alkynyl, (un)saturated cycloalkyl, halogen, hydroxyl, alkoxy, aryloxy, nitro, cyano, amino, substituted amino, hydroxylalkyl, oxo acyl, optionally substituted amino (wherein the substituents are selected from C₁-C₆ alkyl, aryl, aralkyl, or cycloalkyl), aryl, carboxyl, alkaryl, carbamoyl, alkyl, ether, C(═O)NR₅R₆ (wherein R₅ and R₆ are independently selected from hydrogen, alkyl, C₃₋₆ alkenyl, C₃₋₆ alkynyl, aryl, and aralkyl), optionally substituted monocyclic or bicyclic 4-12 membered carbocyclic ring system (wherein the optional substituent(s) is/are selected from alkyl, alkenyl, alkynyl, halogen, hydroxyl, and alkoxy), heteroaryl, heterocyclyl, heteroarylalkyl, or
heterocyclylalkyl];
R₄ is selected from: hydrogen; alkyl; halogen; cyano; carboxy; or C(═O)NRₓR_{y} wherein Rₓ and R_{y} are the same as defined above;
X₁ and X₂ are independently selected from: hydrogen alkyl; alkenyl; alkynyl; cycloalkyl; acyl; aryl; aralkyl; heteroaryl; heterocyclyl; (heteroaryl)alkyl; or (heterocyclyl)alkyl;
Y₁ and Y₂ are independently selected from: hydrogen; alkyl; nitro; cyano, halogen; OR wherein R is the same as defined earlier; SR wherein R is the same as defined earlier; NHR wherein R is the same as defined earlier; COOR′; or COR′ wherein R′ is the same as defined above, or further, Y₁ and X₂, X₁ and Y₂, X₁ and X₂ may together form a ring fused with the ring A containing 3-5 carbon atoms within the ring and having 1-3 heteroatoms selected from N, O or S; and
R₁₃ is alkyl, aryl or heteroaryl;
the method comprising
reacting compounds of Formula XXI with hydroxylamine hydrochloride to give compounds of Formula XXII, 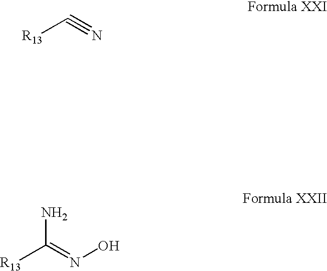
which on reaction with compounds of Formula VI (when Rr is COOH), 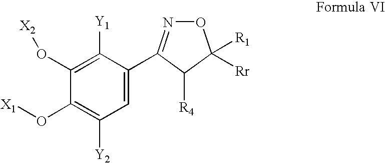
gives compounds of Formula XXIII.

23. (canceled)

24. (canceled)

25. (canceled)

26. (canceled)

27. (canceled)

28. (canceled)
